(19) United States
(12) Patent Application Publication
(10) Pub. No.: US 2003/0207247 A1
(43) Pub. Date: Nov. 6, 2003

(54) Preparation of red blood cells having reduced immunogenicity

(75) Inventors: Adonis Stassinopoulos, Dublin, CA (US); Shruti Mathur, Antioch, CA (US)

Correspondence Address: John W. Tessman, Cerus Corporation, Suite 300, 2525 Stanwell Drive, Concord, CA, 94520, US

(73) Assignee: Cerus Corporation, Concord, CA (US)

(21) Appl. No.: 10/310,618

(22) Filed: Dec. 4, 2002

Related U.S. Application Data
(60) Provisional application No. 60/338,707, filed on Dec. 5, 2001.

(51) Int. Cl. A01N 1/02; G01N 33/567

(52) U.S. Cl. 435/2; 435/7.21

(57) ABSTRACT

Methods are provided for the preparation of an RBC composition which has significantly reduced antigenicity. The methods of preparation of the red cell compositions involve the optimization of reaction conditions, in particular buffering conditions, for attaching antigen masking compounds to the red cells without significantly affecting the function of the red cells. The RBC compositions are of particular use for introduction into an individual in cases where the potential for an immune reaction is high, for example in alloimmunized blood recipients or in trauma situations where the possibility of transfusion of a mismatched unit of blood is higher. The RBC compositions of this invention provide a much lower risk of a transfusion associated immune reaction.

This application claims priority to U.S. Provisional Patent Application Serial No. 60/338,707, filed Dec. 5, 2001; the disclosure of which is hereby incorpaorated by reference.

### FIELD OF THE INVENTION

The present invention relates generally to compositions and methods for reducing risks due to an immune response associated with transfusing cellular compositions from one individual to another. The present invention is primarily concerned with reducing the risk of an immune reaction in the transfusion of a red blood cell (RBC) composition by masking of the antigens on the red cells. The red cell antigens are masked by attaching a non-immunogenic compound to the surface of the red cells.

### BACKGROUND

Blood transfusions are essential in the treatment of patients with anemia, trauma, surgical bleeding and certain inherited disorders. Risks due to an immune reaction in a patient receiving an RBC transfusion include hemolytic transfusion reaction, febrile, non-hemolytic transfusion reaction, transfusion-related acute lung injury, alloimmunization, and graft vs. host disease.

Hemolytic transfusion reactions may be due to reaction of a recipient antibody to an antigen on the donor RBC, often resulting from human error in transfusing a mismatched unit of RBC. Febrile, non-hemolytic transfusion reactions, which account for 90% of transfusion reactions, result from recipient antibodies reacting to an antigen on the donor leukocytes. Transfusion-associated lung injury is believed to be caused by leukocyte antibodies in the donor RBC resulting in the agglutination of recipient leukocytes. Alloimmunization is of greatest risk to patients who require chronic RBC transfusions and results from the patient forming antibodies to minor group antigens on the RBC, which cause reactions in subsequent transfusions (allosensitization). Graft vs. host disease occurs when donor leukocytes engraft and proliferate in the recipient, and then react against tissue antigens of the host.

Elaborate systems of identification and testing are in place to ensure that correctly matched RBC are transfused. However, human performance is a factor in such systems and mistakes are made. A report on transfusion error in New York state indicates ABO incompatible transfusion of 1 in 33,000 units resulting in 3 fatalities [Linden et al., Transfusion 32: 601 (1992)]. Similar error rates have been reported in Great Britain [McClelland et al., BMJ 308:1205 (1994)].

Possible experimental treatments to eliminate the need for ABO matching of blood include enzymatic removal of terminal sugar antigens which convert type A or B RBC to type O [Lenny et al., Transfusion 34:209 (1994), Lenny et al., Biotechnology of blood, Goldstein, J. ed. pp 75-100 (1991)]. Another experimental approach, potentially useful for infusion of RBC into alloimmunized patients, is to mask the RBC antigens by attaching long, flexible hydrophilic molecules such as polyethylene glycol (PEG) to the surface of the RBC creating an essentially non-immunogenic RBC [Scott et al., Proc. Natl. Acad. Sci. USA 94:7566 (1997), U.S. Pat. Nos. 5,908,624, 6,129,912, and 6,312,685, the disclosures of which are hereby incorporated by reference]. The latter patent also discusses improved rheological properties of such modified red cells. The modified red cells have a reduced viscosity at low shear rates, which is beneficial in treating conditions having low blood flow resulting in ischemia. The level of PEG modification required may vary depending on the application. In the case of an RBC that does not need cross matching, a high level of modification may be required in order to mask all antigens. In the case of an allosensitized individual, or for lowering the viscosity, a lesser amount of PEG modification may be sufficient to mask the minor antigens as the blood can be matched for the major antigens. The effects of the above mentioned processes on febrile, non-hemolytic transfusion reaction, transfusion-associated lung injury, and graft vs. host disease is unknown. However, the risk of these three reactions currently can be reduced by leukofiltration of the RBC product.

The search for a blood substitute that is non-immunogenic is ongoing [Ketcham et al., Annals of Emergency Medicine 33:3 pp 326-337 (1999)]. While the above methods demonstrate various means of treating an RBC composition without adversely affecting the blood product, a singular method that produces a fully functioning RBC product which has significantly reduced immunogenicity would be beneficial, unique, and preferable to a blood substitute for most applications as it would have better oxygen transport properties, it would lack mechanism based vasoconstriction side effects found with blood substitutes and it would have a longer in vivo survival time than most blood substitutes. Such an RBC product would be particularly suited for use in alloimmunized patients and in trauma situations where there is no time for crossmatching the blood.

### SUMMARY OF THE INVENTION

The present invention provides new methods for the modification of RBC so that they have significantly reduced immunogenicity and remain suitable for in vivo use. A preferred embodiment of the present invention relates to a method for preparing a composition comprising RBC in which the RBC antigens are substantially masked so that the transfusion of the treated RBC into an antigen mismatched individual would result in a reduced immune reaction compared to the immune reaction of the tranfusion of an untreated RBC composition, where the treated RBC composition is suitable for in vivo use. These results are achieved by the use of an appropriate buffer for the modification of the red cells. Existing methods do not have sufficient buffering capacity or do not provide an appropriate reaction pH to give the desired results. The present invention provides methods of improved masking of red cell antigens involving the reaction of activated antigen masking compounds with the red cells under appropriate buffering conditions that will maintain the desired extracellular pH of approximately 8-10 during the reaction. In addition, the present invention relates to the resulting RBC compositions in which the RBC antigens are substantially masked so as to suitably reduce the risk of eliciting any transfusion reaction associated with an immune response to such antigens.

Methods of the present invention involve the reaction of an activated antigen masking compound to the red cells via an active coupling group. The methods involved are optimal for efficiency of binding to provide sufficient coverage of any antigens without causing significant damage to the red cells so that they remain functional for their intended purpose, e.g. for transfusion. In a preferred embodiment of the invention, the red cell composition is washed at least once with a suitable wash buffer to both remove proteins, which will decrease competitive reaction of the activated antigen masking compound, and provide adequate buffering to enhance the reaction of the antigen masking compound with the red cells. In addition, a suitable reaction buffer is used during the reaction of the activated antigen masking compound with the red cells. Preferred reaction buffers will provide adequate buffering capacity to maintain a pH in the range of 7-10 during the reaction of the antigen masking compound with the red cells. In one embodiment, the reaction buffer comprises a buffer having a concentration of 50-350 mM, also 75-350 mM, also 100-200 mM, or approximately 150 mM, at a pH of 8-10, also 8-9, also 8.5-9, or approximately pH 9. In a preferred embodiment, the wash buffer is the same as the reaction buffer and the red cells are washed at least once with the buffer prior to adding the activated antigen masking compounds. In another embodiment, the wash buffer and reaction buffer provide control of the pH to improve the reaction of antigen masking compound with the red cells yet does not significantly damage the function of the red cells.

### DESCRIPTION OF THE DRAWINGS

FIG. 1 is an exemplary plot of the population maximum fluorescence by flow cytometry for the binding of fluorescent antibody to mPEG modified RBC.

FIG. 2A is an exemplary plot of a standard curve of fluorescence measurement vs. fluorescently labeled activated PEG (FPEG) concentration spiked into red cell ghosts for quantitation of PEG binding to red cells (1.3 billion ghosts per sample).

FIG. 2B is a plot of the FACScan peak (FL1-Height) vs. the number of PEG molecules bound per red cell.

FIG. 3. PEG modification of red cells comparing wash conditions. The relative amount of PEG modification can be assessed by comparison of the Fb1-Height measurement to the positive and negative controls. A higher level of PEG modification is indicated as the FL1-Height of the sample approaches the negative control value.

FIG. 4. PEG modification of red cells comparing buffer strength. The relative amount of PEG modification can be assessed by comparison of the FL1-Height measurement to the positive and negative controls. A higher level of PEG modification is indicated as the FL1-Height of the sample approaches the negative control value.

FIG. 5. Comparison of PEG modification of red cells as a function of the pH of the reaction buffer using fluorescently labeled PEG. Higher FL1-Height indicates greater amount of PEG bound to red cells.

FIG. 6. PEG modification of red cells comparing wash conditions prior to reacting at various pH conditions (indicated as wash condition/reaction condition in the figure, S=saline). The relative amount of PEG modification can be assessed by comparison of the FL1-Height measurement to the positive and negative controls. A higher level of PEG modification is indicated as the FL1-Height of the sample approaches the negative control value.

### DESCRIPTION OF THE INVENTION

The present invention encompasses in vitro or ex vivo methods of treating a composition comprising RBC with a compound comprising a non-immunogenic group having a suitable reactive coupling group, such a compound being referred to as an activated antigen masking compound. The compound is selectively employed to mask RBC antigens by covalently binding to the surface of the RBC resulting in an RBC composition with a significantly reduced immune response when the red cells are reacted with antibodies to the red cell antigens, for example, when infused into an animal of an incompatible blood type. The binding of antigen masking compound to the red cells also reduces the viscosity of the modified red cell at low shear rates. In this application, the level of antigen masking compound bound per red cell can be lower, as the viscosity reduction at low shear rates can be achieved with less antigen masking compound bound than required for antigen masking of the red cells. Such low viscosity red cells can be used to treat ischemic conditions, such as those resulting from stroke, myocardial infarction, sickle cell anemia, and other conditions relating to vascular occlusion (see U.S. Pat. No. 6,312,685). Additional diseases that can be treated include angina, critical limb ischemia, cerebral vasospasm, and subarrachnoid hemorrhage. The present invention generally relates to new methods for preparing compositions comprising RBC that have significantly reduced immunogenicity, remain functional and are suitable for in vivo use, e.g. for transfusion. The invention also relates to methods of use of the resulting RBC compositions such as for transfusion using techniques known in the art. The methods of the present invention are optimized to give efficient reaction of the antigen masking compound with the red cells while maintaining adequate function of the red cells. It is important to optimize the pH of the reaction of the antigen masking compounds with the red cells. The pH optimization involves controlling the extracellular pH of the solution while maintaining a suitable intracellular pH of the red cells to avoid or reduce any damage to the red cell function. Preferred methods provide an extracellular pH of approximately 8-10, preferably 8.5-9.5, while maintaining the intracellular pH between approximately 7-8.

In some embodiments a quencher is included which would effectively react with any excess compounds of the present invention. Additionally, a process of removing any excess compounds, products of the compound's reaction with a quencher, side products of the process, and the quencher itself are contemplated in the present invention.

In vivo use of a material or compound is defined as introduction of the material or compound into a living individual. For example, the transfusion of a blood product into an individual in need of a transfusion would be considered an in vivo use of the blood product. An individual, as defined herein, is a vertebrate, preferably a mammal, including domestic animals, sport animals, and primates, including humans.

In vitro use of a material or compound is defined as a use of the material or compound outside a living individual, where typically neither the material nor compound is intended for reintroduction into a living individual. Ex vivo use of a compound is defined as using a compound for treatment of a biological material outside a living individual, where the treated biological material is intended for use inside a living individual. For example, removal of blood from a human and introduction of a compound into that blood to inactivate pathogens is defined as an ex vivo use of that compound if the blood is intended for reintroduction into that human or another human. Reintroduction of the human blood into that human or another human would be in vivo use of the blood, as opposed to the ex vivo use of the compound. If the compound is still present in the blood when it is reintroduced into the human, then the compound, in addition to its ex vivo use, is also introduced in vivo.

The compositions considered in the present invention are considered to be functional if certain in vitro and in vivo properties are similar to the properties of a sample that is not treated by the methods of the present invention. In addition, there are certain blood banking standards that will ideally be met by compositions of the present invention. To be suitable for in vivo use, the compositions must also exhibit low levels of toxicity, as measured for example by gene toxicity, animal studies and Ames mutagenicity assays.

The present invention contemplates the antigen masking or immune masking of RBC. RBC comprise several antigenic determinants on their surface that might cause an immune response. For example, the immune system of a recipient of an RBC transfusion may recognize certain antigens on the transfused RBC as foreign and mount an immune response to the RBC. Masking of these antigens involves the modification or hiding of these antigens so that any immune response elicited in the recipient is significantly reduced. In one embodiment, the antigens are masked so that they are no longer accessible to or recognized by the immune system of the recipient. Certain compounds may be linked to the RBC surface such that the antigens on the RBC surface are hidden or masked by the compound. In addition, these compounds that can be linked to the RBC surface may have a structure that is not itself recognized by an immune system, i.e. these compounds are non-immunogenic or non-antigenic. By masking antigens in this manner, an immune response elicited in the recipient by the transfused RBC is significantly reduced. RBC antigens are considered to be substantially masked when the treated RBC have significantly reduced reactivity toward antibodies that bind specific RBC antigens when compared to the binding of an untreated RBC. This reduced immunogenicity can be readily measured using in vitro antibody binding assays or in vitro measurements of the amount of modification of the RBC. Further, the treated red cells can be tested in vivo to assess whether they provide a reduction or elimination of an immune response in a recipient.

Quenchers as used herein refer to compounds that are capable of reacting with activated antigen masking compounds encompassed by the present invention. The quencher may serve to react with any excess activated antigen masking compound, after the compound has sufficiently reacted with its intended target on the surface of the red cells.

I. Methods and Compounds for Masking Immunogenicity of the Red Blood Cells

The present invention further contemplates treating a red cell composition with a compound that will covalently bind to the surface of the RBC and substantially mask any antigens present on the cells. A red cell composition is considered to be any composition comprising RBC. Such compositions typically comprise red cells ranging in hematocrit from about 1% to about 65% or higher, such as approximately 20-95%, also 30-95%, also 40-95%, also 50-95%, preferably 60-95%, or 60-80%. In one embodiment the red cells are prepared as a red cell concentrate having a hematocrit of approximately 80-95%. The red cell concentrate can either be washed with an appropriate wash solution or it can be diluted to the desired hematocrit for reacting with activated antigen masking compound. In one embodiment, red cells are collected by standard blood banking techniques and the red cells are centrifuged at 4° C. at approximately 4100×g for approximately 6 minutes and the supernatant plasma is removed to prepare the red cell concentrate. In another embodiment, it is contemplated that prior to treating with an activated antigen masking compound, the RBC composition may be washed to reduce the level of plasma proteins in order to decrease competitive processes that may affect the level of antigen masking of the RBC. This wash may be done by adding at least an equal volume of wash solution to a red cell composition, mixing, centrifuging and removing the supernatant. In one embodiment, the red cell composition is washed with a volume of wash solution equal to the volume of the red cell composition. In another embodiment, the wash solution is at least 2×, 5×, 10×, or 20× the volume of the red cell composition. In one embodiment, the wash step may be done more than once. In a preferred embodiment, the red cells are first prepared as a red cell concentrate prior to adding the wash solution. In one embodiment, a volume of wash solution equal to the volume of a red cell concentrate is mixed with the red cell concentrate, centrifuged as described above and the supernatant removed to prepare a washed red cell concentrate. The washed red cell concentrate may be washed again or may be mixed with a reaction solution and an activated antigen masking compound. The reaction solution and activated antigen masking compound can be added to the desired hematocrit for the reaction. Typically, the activated antigen masking compound is dissolved in the reaction solution and this is added to the washed red cell concentrate. If the desired hematocrit for the reaction is that of a red cell concentrate (i.e. 80-95%), the activated antigen masking compound may be dissolved directly in the washed red cell concentrate. Alternatively, the desired concentration of activated antigen masking compound can be dissolved in reaction solution and the red cell concentrate can be mixed with a suitable volume of this mixture, centrifuged and the supernatant removed to the desired hematocrit in order to provide an 80-95% hematocrit solution with the desired concentration of activated antigen masking compound. The activated antigen masking compounds do not penetrate the red cell membranes, therefore the concentration of compound is determined based only on the extracellular volume of the solution.

In addition to the removal of proteins, washing of the RBC with a buffer of the appropriate pH reduces (quenches) the buffering capacity of the hemoglobin inside the RBC or the total buffering capacity of the RBC. One aspect of the invention is that methods are provided to achieve adequate buffering even at the highest hematocrit levels, such as 50-95%. The reaction of activated antigen masking compounds at these higher hematocrit levels is found to be more efficient. Reaction of red cells with a 5 kDa mPEG-SPA-NHS at the same extracellular concentration results in the same level of antigen masking of the red cells at 40, 60 or 80% hematocrit as determined by FACScan analysis of fluorescent labeled antibody binding to the cells. As hematocrit increases, a lesser amount of the mPEG is used with a higher number of red cells. Since level of antigen masking does not change, it is far more cost effective to react at the higher hematocrit. Therefore, it is preferred for this invention to carry out the reaction at a hematocrit of greater than 50%, also greater than 60%, or at approximately 60-95%. A detailed discussion of the reaction of antigen masking compounds with red cells at high hematocrit can be found in concurrently filed U.S. Provisional Application titled “Methods for Antigen Masking of RBC with High Efficiency”. The RBC utilize metabolic activities, for example the use of hydrogen ion pumps and the consumption of ATP, to maintain pH stasis. The reaction of the compounds of the present invention with the surface of the red cells can be optimized by using a suitable buffer to adjust the pH of the solution to a range that is optimal for the antigen masking of the RBC. A preferred buffer will be at a pH of approximately 7-11, preferably approximately 8-10, also 8.5-9.5, also 8.5-9 or approximately 9. The buffer will have a sufficient buffering capacity to maintain an appropriate pH during the reaction. The buffering capacity is related to both optimal buffer range of the buffer and the concentration of buffer. Preferred buffers will comprise a buffer at a concentration ranging from approximately 50 mM to 350 mM, also 75-350 mM, also 100-200 mM, or approximately 150 mM and will be at a pH near it's optimal buffering capacity (i.e. the buffer should have a pKa at or near the desired buffering pH). The washing of the RBC with such a buffer reduces the gradual alteration of the pH from the optimal higher than physiologic value of approximately 8-9 to the physiological value of approximately 7. This lowering of the pH results in the reduction of the reaction rate of the activated antigen masking compound with the RBC surface and the reduction of the amount of modification achieved before the activated antigen masking compound decomposes. The buffering of the pH achieved by washing will increase the efficiency of the antigen masking reaction. In another embodiment, the buffering to increase the efficiency of the antigen masking reaction will not significantly affect the function of the RBC. The buffering can be optimized to provide adequate maintenance of the extracellular pH at higher pH values, which increases the reaction of the antigen masking compound with the red cells, while maintaining a suitable lower intracellular pH value, which is important to maintain the red cell function. In a preferred embodiment, the buffers used in the current invention will maintain an intracellular pH of approximately 7-9, preferably approximately 7-8. The washing conditions prior to addition of the activated antigen masking compound are adjusted to provide better reaction conditions. In one embodiment, the red cell composition is washed with a wash buffer prior to adding the antigen masking compound. In one embodiment, the washed red cells are mixed with a reaction buffer and the antigen masking compound. In one embodiment, the antigen masking compound is dissolved in the reaction buffer prior to mixing with the red cell composition. In one embodiment, the reaction buffer is the same as the wash buffer. Once the reaction with the antigen masking compound is complete, the red cells can be further washed to provide an adequate pH for long term storage, such as physiological pH (i.e approximately 7).

In addition to providing the appropriate pH for improved reactivity of the activated antigen masking compounds with the red cells, the buffers of use in the present invention have adequate buffering capacity to counter the buffering capacity of the hemoglobin in the red cells or the total buffering capacity of the red cells. Preferred buffers of the present invention, when used at hematocrits in the range of 20-95%, also 30-95%, also 40-95%, also 50-95%, preferably 60-95%, or 60-80% will be at a concentration of approximately 50 mM to 350 mM, preferably approximately 75 mM to 350 mM, more preferably approximately 100 mM to 200 mM, most preferably approximately 150 mM. In a preferred embodiment, buffers are used at a hematocrit range of 60-95%, preferably 60-80%, wherein the buffers are at approximately 50-350 mM, preferably 75-350 mM, more preferably 100-200 mM, or approximately 150 mM. A higher hematocrit generally requires a higher concentration of buffer to achieve adequate pH during the reaction. Examples of buffers that are useful for the present invention to provide adequate buffering in the range of approximately pH 7-10 include, but are not limited to, the following, where the pKa (at 25° C.) and useful pH range are indicated after the buffer acronym. [(2-Hydroxy-1,1-bis[hydroxymethyl]ethyl)amino]-1-propanesulfonic acid (TAPS, pKa 8.40, pH 7.7-9.1), 2-Amino-2-methyl-1,3-propanediol (AMPD, pKa 8.80, pH 7.8-9.7), N-tris-(Hydroxymethyl)methyl-4-aminobutanesulfonic acid (TABS, pKa 8.90, pH 8.2-9.6), 3-([1,1-Dimethyl-2-hydroxyethyl]amino)-2-hydroxypropanesulfonic acid (AMPSO, pKa 9.00, pH 8.3-9.7), N-(2-Hydroxyethyl)piperazine-N′-(2-ethanesulfonic acid) (HEPES, pKa 7.48, pH 6.8-8.2), 3-(Cyclohexylamino)-2-hydroxy-1-propanesulfonic acid (CAPSO, pKa 9.60, pH 8.9-10.3), 2-(N-Cyclohexylamino)ethanesulfonic acid (CHES, pKa 9.30, pH 8.6-10.0), phosphate buffers, and Triethanolamine (pKa 7.8, 7.3-8.3). Buffers for use in the present invention have a pKa (at 25° C.) that is within 1.0, preferably 0.7, preferably 0.5 pH units of the desired pH of the buffering solution. Preferred buffers for use in the invention have a pKa of approximately 8.0-10.0, preferably 8.3-9.7, more preferably 8.5-9.5. In one embodiment, the solutions used in the processing of the red cells further comprise an additive that helps to reduce hemolysis during the process. Such additives include dextrose and L-carnitine. In one embodiment, the buffer further comprises 50-300 mM dextrose, preferably approximately 75-200 mM dextrose, preferably approximately 100 mM dextrose. In one embodiment, the buffer further comprises L-carnitine at approximately 2-100 mM, preferably 2-10 mM, more preferably approximately 5 mM. In one embodiment, the buffer further comprises both 50-300 mM dextrose, preferably approximately 75-300 mM dextrose, or approximately 100 mM dextrose and L-carnitine at approximately 2-100 mM, preferably 2-10 mM, more preferably approximately 5 mM. A preferred buffer for use in the present invention comprises CHES buffer. In one embodiment, the buffering solution for use in the present invention comprises approximately 150 mM CHES and approximately 50 mM sodium chloride (NaCl), adjusted to a pH of approximately 9.0. A preferred buffering solution comprises approximately 150 mM CHES, 100 mM dextrose, and 5 mM L-carnitine at a pH of 9.0. It is necessary that the buffer compositions used are compatible with the RBC function. They should not cause significant lysis or alteration of the RBC properties other than the pH change. More preferably, the buffers used to change the pH on the extracellular domain of the red cells will not significantly change the intracellular pH of the red cells. Preferred buffers enhance the reactivity of the attachment groups on the red cell surface so that they are more reactive with the activated antigen masking compounds.

Appropriate buffering conditions for both reaction of activated antigen masking compound and for long term storage may also be achieved by addition of a resin material to alter the buffering capacity of the red cell solution. A resin is defined as any solid material that can achieve the change of pH without being dissolved in the solution and encompasses man-made or naturally occurring materials, such as solid minerals. Such a resin could reduce or eliminate the washing requirements discussed above in order to achieve a suitable pH for the reaction of the compounds with the red cells. In one embodiment, the resins are retained in a pouch material of suitable pore size to allow for equilibration of the pH without allowing contact of the red cells with the resin material. In this embodiment, the resin material can be readily removed from contact with the red cell solution as needed. In addition, the red cell material can be removed from contacting a resin material that provides optimal pH for reaction of the antigen masking compound and contacted with another resin which returns the red cell solution to physiological pH or other appropriate conditions for long term storage.

In order to provide adequate pH for reaction of the activated antigen masking compounds with the red cells, it is possible to use either a strong cation exchange resin or an anion exchange resin that is equilibrated with a base. A strong cation exchange resin would raise the pH by adsorbing hydrogen ions. Examples of such resins include, but are not limited to, polystyrene based porous adsorbents that have either a sulfonic acid or carboxylic acid functional group. An anion exchange resin that is equilibrated with a base (e.g. a weak acid) would elevate the pH. While not intended to limit the mechanism of such a resin, the resin may be equilibrated with, for example, phosphate ion (PO₄⁻²) or hydroxide ion (OH⁻) which would be released by the resin and bind with protons, resulting in a higher pH. The approach using an anion exchange resin would also serve to eliminate certain proteins from the solution. Such proteins would have a net negative charge at the elevated pH and would bind and be removed by the resin. This would eliminate such proteins from competing with the red cell surface for reaction with the activated antigen masking compound. In order to bring the pH back to physiological levels after reacting with the activated antigen masking compound, the solution can be removed from contacting the resins discussed above and either washed in an appropriate buffer, or contacted with another resin material. For this purpose, it is possible to use either a strong anion exchange resin or a cation exchange resin that is equilibrated with hydrogen ions. A strong anion exchange resin would lower the pH by absorbing hydroxide ion while the cation exchange resin would release the hydrogen ions. An example of processing with such resins would be to prepare a unit of red cell concentrate and adjusting the hematocrit using an appropriate buffer. The unit is transferred to another blood bag containing a suitable ion exchange resin, such as 10 g of IRA-400 OH resin (Supelco, Bellefonte, Pa.). The resin is contained in a mesh pouch that keeps the red cells from contacting the resin material yet allows pH equilibration to the desired pH. The activated antigen masking compound is then added to the bag to the desired concentration and reacted. After the reaction, the red cell solution is transferred to another bag containing a suitable ion exchange resin in a pouch in order to bring the pH back to physiological levels. Alternatively, instead of contacting with another resin, the red cells can be washed and stored in a suitable storage solution.

Without intending to be limited to any particular mechanism of action of the present invention, activated antigen masking compounds for covalent binding to RBC in order to mask antigens will comprise a non-immunogenic group and a coupling group. In one embodiment, upon linking the non-immunogenic group to a target molecule, a portion of the coupling group remains. In another embodiment, the coupling group is such that the non-immunogenic group is directly linked to a target molecule, leaving no portion of the coupling group.

In one embodiment, the non-immunogenic portion of the compound is polyethylene glycol (PEG) or a derivative of PEG. A thorough discussion of the use of PEG technology can be found in PCT publication 95/06058, the disclosure of which is hereby incorporated by reference. Further discussion of the use of PEG for modification of red cells can be found in U.S. Pat. Nos. 5,908,624, 6,129,912, and 6,312,685. Additional compounds suitable for the present invention would comprise other non-immunogenic groups capable of blocking recognition of antigens on an RBC surface when covalently attached to the RBC surface. Such non-immunogenic groups include, but are not limited to, polyalkylene glycols (such as PEG, polypropylene glycol, mixed polypropylene-polyethylene glycols, i.e. poloxamers), polyalkylene glycol derivatives (such as methoxy(polyethyleneglycol), mPEG), polysaccharides (such as dextrans, cellulosics, Ficoll, and arabinogalactan), and hydrophilic, synthetic polymers such as polyurethanes. Copolymers of PEG with other hydrophilic materials, such as glycerols, polysaccharides, or polyurethanes, are also useful. Polyoxyethylated glycerol (POG) is an example of such a copolymer.

Preferred activated antigen masking compounds comprise PEG and derivatives of PEG attached to a suitable coupling group. Such PEG compounds are also referred to as activated PEG compounds and have the general formula Cp-(OCH₂CH₂)ₙ—OH wherein n is greater than or equal to 3 and Cp represents a coupling group which reacts with terminal thiol or amine groups on an RBC surface to covalently link the non-immunogenic group to the RBC. The molecular weight can vary up to approximately 200 kiloDalton (kDa) or more. Preferred derivatives have a molecular weight range of 2-40 kDa, in some embodiments 5-40 kDa, 10-40 kDa, 15-40 kDa, 20-40 kDa, 20-30 kDa, or 20-25 kDa. Derivatives wherein the end groups are modified include, but are not limited to, PEG ethers (e.g.Cp-(OCH₂CH₂)ₙ—OR, such as Cp-(OCH₂CH₂)ₙ—OCH₃ (mPEG)), PEG esters (e.g. Cp-(OCH₂CH₂)ₙ—OOCR, such as Cp-(OCH₂CH₂)ₙ—OOC(CH₂)₁₄ CH₃), PEG amides (e.g. Cp-(OCH₂CH₂)ₙ—OOC(CH₂)₇CONHR), PEG amines (e.g. Cp-(OCH₂CH₂)ₙ—NH₂,), PEG acids (e.g. Cp-(OCH₂CH₂)ₙ—OCH₂COOH), PEG aldehydes (e.g. H—(OCH₂CH₂)ₙ—OCH₂CHO), and electrophilic derivatives such as halogenated PEG (e.g. H—(OCH₂CH₂)ₙ—Br. The preferred derivatives of the present invention are those of mPEG. Another embodiment of the present invention contemplates branched PEG and branched PEG derivatives in which PEG arms are linked giving multi armed branched molecules. A further embodiment of branched PEG derivatives includes derivatives which can form crosslinks when bound to the red cell surface. Such branched PEG compounds bound to red cells are able to link with intermolecular red cell bound PEG forming crosslinks. Such crosslinks may provide a protective network around the red cell and be more effective at antigen masking the red cells. Generally, the branched PEGs for crosslinking would require at least another reactive electrophilic center and the use of a multivalent nucleophile such as a polyamine or a protein molecule that contains multiple nucleophiles. Reaction of the first electrophilic center of the PEG will result in attachement of the PEG to the RBC, while the other reactive center would be in a position that allows the reaction with one of the valences of the polynucleophile. Since the polynucleophile can react with multiple PEG centers it will generate a crosslinked matrix above the RBC (e.g. see U.S. Pat. No. 6,129,912). Another embodiment of the present invention contemplates a mixture of two or more of the above mentioned types of compounds. A further embodiment contemplates a mixture of two or more of these types of activated PEG and PEG derivative compounds in which the coupling group is targeted to different nucleophiles on the RBC surface. For example, a mixture of activated mPEGs may be used wherein one mPEG may be activated with a coupling group that preferably reacts with an amine group while another mPEG may be activated with a coupling group that preferably reacts with a thiol group.

In one embodiment, the coupling group for linking the non-immunogenic group to the RBC comprises a reactive group which reacts with terminal thiol or amine groups on the RBC surface. Examples include, but are not limited to, sulphonate esters, substituted triazines, N-hydroxysuccinimide esters, anhydrides, activated carbonates, substituted phenyl carbonates, oxycarbonylimidazoles, maleimides, aldehydes, glyoxals, carboxylates, vinyl sulphones, epoxides, mustard, mustard equivalents, isocyanates, isothiocyanates, disulphides, acrylates, allyl ethers, silanes, and cyanate esters. Mustards are herein defined as including mono or bis-(haloethyl)amine groups, and mono haloethylsulfide groups. Mustard equivalents are herein defined as groups that react by a mechanism similar to the mustards (i.e. by forming reactive intermediates such as aziridinium or aziridine complexes and sulfur analogs of these complexes). Examples of such mustard equivalents includes aziridine derivatives, mono or bis-(mesylethyl)amine groups, mono mesylethylsulfide groups, mono or bis tosylethylamine groups, and mono tosylethylsulfide groups. Other possible coupling groups are selected from 2,2,2-trifluoroethanesulphonate, pentafluorobenzenesulphonate, fluorosulphonate, 2,4,5-trifluorobenzenesulphonate, 2,4-difluorobenzenesulphonate, 2-chloro-4-fluorobenzenesulphonate, 3-chloro-4-fluorobenzenesulphonate, 4-amino-3-chlorobenzenesulphonate, 4-amino-3-fluorobenzenesulphonate, o-trifluoromethylbenzenesulphonate, m-trifluoromethylbenzenesulphonate, 2-trifluoromethoxybenzenesulphonate, 4-trifluoromethoxybenzenesulphonate, 5-fluoro-2-methylbenzenesulphonate, 4,6-dichlorotriazine, 6-chlorotriazine, N-hydroxysuccinimidyl succinate, N-hydroxysuccinimidyl glutarate, N-hydroxysuccinimidyl succinamide, N-hydroxysuccinimidylalkanedioicamides, N-hydroxysuccinimidyl derivatives of carboxymethylated polymers, N-hydroxysuccinimidyl esters of amino acids, succinimidylcarbonate, succinate mixed anhydride, succinic anhydride, 2,4,5-trichlorophenol, trichlorophenyl carbonate, nitrophenyl carbonate, 4-nitrophenol, cyanuric chloride, maleimide, N-substituted maleimide, acetaldehyde, propionaldehyde and chemically equivalent sulfur analogs, glyoxal, phenylglyoxal, acrylate, methacrylate, fluoro substituted phenyl esters and their sulfur analogs, fluoro substituted ethyl esters and fluoro substituted ethanethiol esters. In another embodiment, the coupling group is a halogen atom, preferably iodide, bromide or chloride. In some instances the reactivity of the group may be increased through the use of a catalyst. This catalyst may be an enzyme, such as transglutaminase, or a man-made or naturally occurring compound, such as iodide used as a nucleophilic catalyst, used in substoichiometric or stoichiometric amounts.

A preferred embodiment of an activated non-immunogenic compound is 2,2,2-trifluoroethanesulphonylmonomethoxy polyethylene glycol (Tresyl mPEG, or TmPEG). Other preferred embodiments of an activated non-immunogenic compound are Methoxy(polyethyleneglycol)-succinimidyl propionate (mPEG-SPA-NHS), Methoxy(polyethyleneglycol)-succinimidyl butanoate (mPEG-SBA-NHS), Methoxy(polyethyleneglycol)-succinimidyl carbonate (mPEG-SC), N-3-[Methoxy(polyethyleneglycol)]-oxo-aminopropionate N-hydroxysuccinimide ester (mPEG-βA-NHS), N-6-[Methoxypoly(ethyleneglycol)]-oxo-aminocaproate N-hydroxysuccinimide ester (mPEG-6AC-NHS), N-5-[Methoxypoly(ethyleneglycol)]-oxo-aminovalerate N-hydroxysuccinimide ester (mPEG-5AV-NHS), N-4-[Methoxypoly(ethyleneglycol)]-oxo-aminobutyrate N-hydroxysuccinimide ester (mPEG-4AB-NHS), N-6-[Methoxypoly(ethyleneglycol)]-oxo-aminocaproate pentafluorophenyl ester (mPEG-6AC-PFP), N-6-[Methoxypoly(ethyleneglycol)]-oxo-aminocaproate 2,2,2-trifluoroethyl ester (mPEG-6AC-TFE), N-6-[Methoxypoly(ethyleneglycol)]-oxo-aminocaproate ethyl ester (mPEG-6AC-OEt), N-6-[Methoxypoly(ethyleneglycol)]-oxo-aminocaproate pentafluorobenzenethio ester (mPEG-6AC-PFT), N-6-[Methoxypoly(ethyleneglycol)]-oxo-aminocaproate 2,3,5,6-tetrafluorobenzene thio ester (mPEG-6AC-TFT), N-6-[Methoxypoly(ethyleneglycol)]-oxo-aminocaproate 4-fluorobenzenethio ester (mPEG-6AC-4FTP), N-6-[Methoxypoly(ethyleneglycol)]-oxo-aminocaproate 2,2,2-trifluoroethylthio ester (mPEG-6AC-TFET), and N-6-[Methoxypoly(ethyleneglycol)]-oxo-aminocaproate ethylthio ester (mPEG-6AC-SEt), the structures of which are as given below, where n is greater than or equal to 1, preferably greater than or equal to 3.

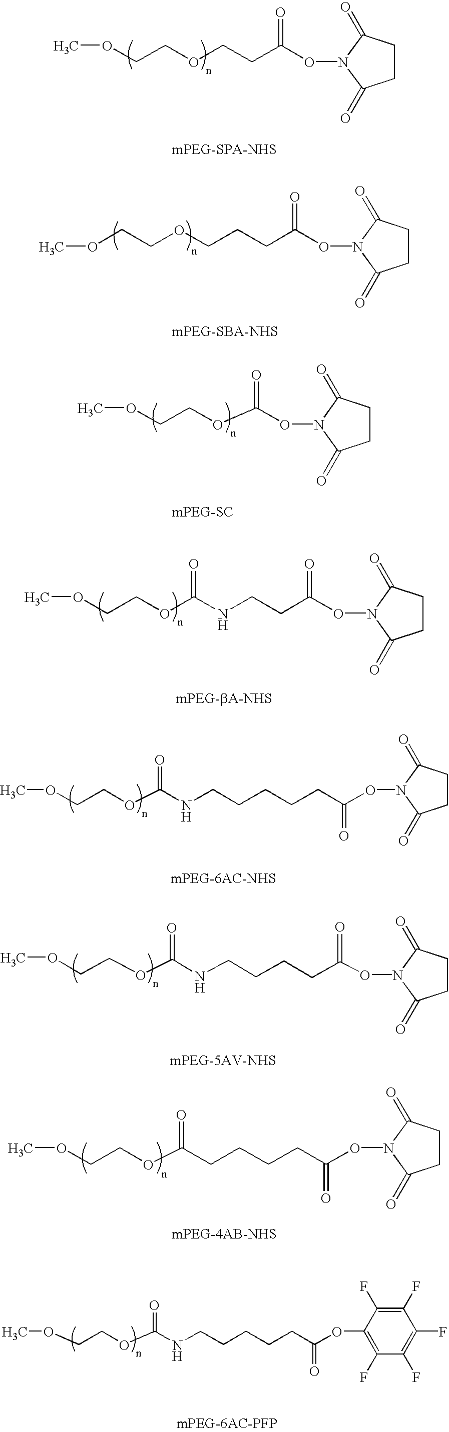

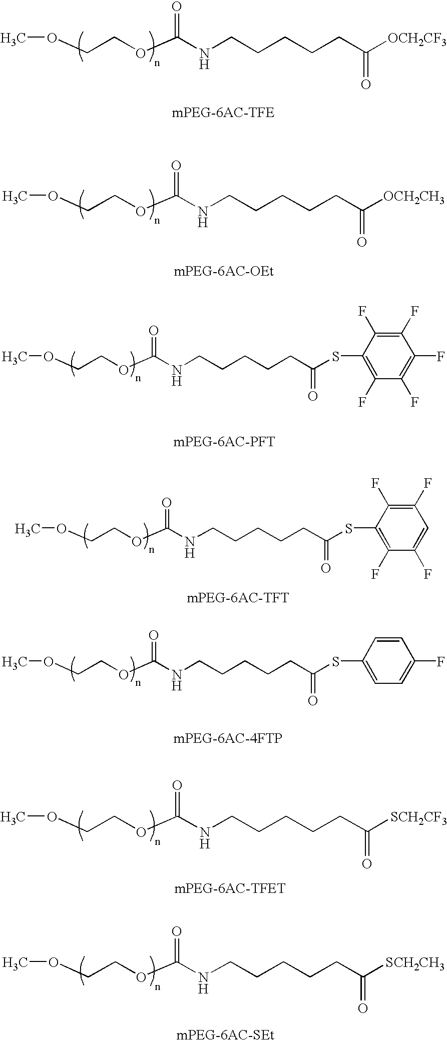

In one embodiment, the present invention encompasses the modification of red cells with an antigen masking compound, wherein an activated antigen masking compound is reacted with a red cell composition mixed with a reaction solution comprising a buffer at a concentration of approximately 50-350 mM, preferably 75-350 mM, preferably 100-200 mM, more preferably approximately 150 mM, wherein the buffer is at a pH of approximately 8-10, preferably 8.5-9.5, more preferably 8.5-9, most preferably approximately 9. In a preferred embodiment, the red cell composition is washed prior to reacting with the activated antigen masking compound, wherein the wash solution comprises a buffer at a concentration of approximately 50-350 mM, preferably 75-350 mM, preferably 100-200 mM, more preferably approximately 150 mM, wherein the buffer is at a pH of approximately 8-10, preferably 8.5-9.5, more preferably 8.5-9, most preferably approximately 9. In one embodiment, the wash solution and reaction solution are the same. In one embodiment, the red cells are washed by adding the wash solution to a red cell concentrate. In one embodiment, the volume of wash solution used is greater than or equal to the volume of the red cell concentrate. In one embodiment the reaction solution is added to a red cell concentrate. In some embodiments, the reaction is done at a hematocrit of 20-95%, also 30-95%, also 40-95%, also 50-95%, preferably 60-95%, or 60-80%. In one embodiment, the activated antigen masking compound is dissolved in the reaction solution prior to adding to the red cell composition. In one embodiment, the reaction solution is added to the red cell composition and the activated antigen masking compound is dissolved in this mixture. In some embodiments, the activated antigen masking compound has a molecular weight of 2-40 kDa, also 5-40 kDa, 10-40 kDa, 15-40 kDa, 20-40 kDa, 20-30 kDa, or 20-25 kDa. In a preferred embodiment, the activated antigen masking compound has a molecular weight of 5-40 kDa, preferably 15-40 kDa, preferably 20-40 kDa, more preferably 20-30 kDa. The molar concentration of activated antigen masking compound that is most effective depends to some extent on the size of the antigen masking compound used. Generally, the larger compounds require lower molar concentrations than the smaller compounds. Since the antigen masking compounds do not penetrate the red cell membranes, the concentrations are based on the extracellular volume of the samples being reacted. Generally, activated antigen masking compounds can be used over a range of approximately 1-50 mM. For activated antigen masking compounds in the range of 15-40 kDa, the concentration used may be in the range of 1-30 mM, also 1-20 mM, also 2-15 mM or 2-10 mM. Antigen masking compounds in the range of 2-15 kDa can be used at higher concentrations, such as 5-50 mM, also 5-30 mM, 5-25 mM. In one embodiment, the activated PEG is in the range of 20-30 kDa at a concentration of 1-20 mM, also 2-10 mM. In a further embodiment, the activated antigen masking compound is a PEG derivative. In one embodiment, the activated antigen masking compound is a methoxy(polyethyleneglycol) (mPEG). In one embodiment the activated mPEG is selected from the group consisting of mPEG-SPA-NHS, mPEG-SBA-NHS, mPEG-SC, mPEG-βA-NHS, mPEG-6AC-NHS, mPEG-5AV-NHS, mPEG-4AB-NHS, mPEG-6AC-PFP, mPEG-6AC-TFE, mPEG-6AC-OEt, mPEG-6AC-PFT, mPEG-6AC-TFT, mPEG-6AC-4FTP, mPEG-6AC-TFET, and mPEG-6AC-SEt. In a preferred embodiment, the activated mPEG is selected from the group consisting of mPEG-6AC-NHS, mPEG-5AV-NHS, and mPEG-4AB-NHS. In a more preferred embodiment, the activated mPEG is mPEG-6AC-NHS.

In one embodiment, the present invention encompasses a method of preparing a modified red cell composition comprising a) providing i) a red cell composition, ii) an activated antigen masking compound, iii) a wash solution, and iv) a reaction solution; b) washing the red cell composition with the wash solution; c) mixing the washed red cell composition with the reaction solution and the activated antigen masking compound to provide a reaction mixture; and d) incubating the reaction mixture so that the activated antigen masking compound covalently binds to the red cell surface. In one embodiment, the mixture of step c is at a hematocrit of 20-95%, also 30-95%, also 40-95%, also 50-95%, preferably 60-95%, or 60-80%. In one embodiment, the incubation is done at a temperature ranging from 4-40° C., preferably 20-25° C. In another embodiment, the incubation is for approximately 5-240 minutes, also 10-120 minutes, preferably 30-120 minutes, more preferably approximately 60 minutes. In one embodiment, the red cell composition is a red cell concentrate. In one embodiment, the red cells are washed by adding the wash solution to the red cell concentrate. In one embodiment, the volume of wash solution used is greater than or equal to the volume of the red cell concentrate. In one embodiment, the activated antigen masking compound has a molecular weight of approximately 2-40 kDa, in some embodiments 5-40 kDa, 10-40 kDa, 15-40 kDa, 20-40 kDa, 20-30 kDa, or 20-25 kDa. In a preferred embodiment, the activated antigen masking compound has a molecular weight of 5-40 kDa, preferably 15-40 kDa, preferably 20-40 kDa, more preferably 20-30 kDa. In one embodiment, the activated antigen masking compound is at a concentration of approximately 1-50 mM. For activated antigen masking compounds in the range of 15-40 kDa, the concentration used may be in the range of 1-30 mM, also 1-20 mM, also 2-15 mM or 2-10 mM. Antigen masking compounds in the range of 2-15 kDa can be used at higher concentrations, such as 5-50 mM, also 5-30 mM, 5-25 mM. In one embodiment, the activated antigen masking compound is in the range of 20-30 kDa at a concentration of 1-20 mM, also 2-10 mM. In a further embodiment, the activated antigen masking compound is a PEG derivative. In one embodiment, the activated antigen masking compound is an mPEG. In one embodiment the activated MPEG is selected from the group consisting of mPEG-SPA-NHS, mPEG-SBA-NHS, mPEG-SC, mPEG-βA-NHS, mPEG-6AC-NHS, mPEG-5AV-NHS, mPEG-4AB-NHS, mPEG-6AC-PFP, mPEG-6AC-TFE, mPEG-6AC-OEt, mPEG-6AC-PFT, mPEG-6AC-TFT, mPEG-6AC-4FTP, mPEG-6AC-TFET, and mPEG-6AC-SEt. In a preferred embodiment, the activated mPEG is selected from the group consisting of mPEG-6AC-NHS, mPEG-SAV-NHS, and mPEG-4AB-NHS. In a more preferred embodiment, the activated mPEG is mPEG-6AC-NHS.

In one embodiment, the wash solution comprises a buffer having a concentration ranging from approximately 50 to 350 mM, preferably 75-350 mM, preferably 100-200 mM, preferably approximately 150 mM. In one embodiment, the wash solution comprises a buffer having a pH in the range of approximately 8-10, preferably 8.5-9.5, more preferably 8.5-9, more preferably approximately 9. In another embodiment, the wash solution comprises a buffer having a pKa of approximately 8.5-9.5, preferably approximately 9. In a further embodiment, the buffer is selected from the group consisting of TAPS, AMPD, TABS, AMPSO, CAPSO, and CHES. In a preferred embodiment, the buffer is CHES. In another embodiment, the wash solution comprises dextrose. In a further embodiment, the dextrose is at a concentration of 50-300 mM, preferably approximately 75-200 mM, preferably approximately 100 mM. In a further embodiment, the wash solution comprises L-carnitine, preferably at a concentration of 2-100 mM, preferably 2-10 mM, more preferably approximately 5 mM. In one embodiment, the wash solution comprises 150 mM CHES, 100 mM dextrose and 5 mM L-carnitine. In one embodiment, the reaction solution comprises a buffer having a concentration ranging from approximately 50 to 350 mM, preferably 75-350 mM, preferably 100-200 mM, preferably approximately 150 mM. In one embodiment, the reaction solution comprises a buffer having a pH in the range of approximately 8-10, preferably 8.5-9.5, more preferably 8.5-9, more preferably approximately 9. In another embodiment, the reaction solution comprises a buffer having a pKa of approximately 8.5-9.5, preferably approximately 9. In a further embodiment, the buffer is selected from the group consisting of TAPS, AMPD, TABS, AMPSO, CAPSO, and CHES. In a preferred embodiment, the buffer is CHES. In another embodiment, the reaction solution comprises dextrose. In a further embodiment, the dextrose is at a concentration of 50-300 mM, preferably approximately 75-200 mM, preferably approximately 100 mM. In a further embodiment, the wash solution comprises L-carnitine, preferably at a concentration of 2-100 mM, preferably 2-10 mM, more preferably approximately 5 mM. In one embodiment, the reaction solution comprises 150 mM CHES, 100 mM dextrose and 5 mM L-carnitine. In one embodiment, the wash solution and reaction solution are the same.

In one embodiment, the present invention encompasses a method of preparing a red cell composition comprising a) providing i) a red cell concentrate, ii) an activated antigen masking compound, and iii) a reaction solution; b) washing the red cell concentrate with the reaction solution to provide a washed red cell concentrate; c) mixing the washed red cell concentrate with the reaction solution and the activated antigen masking compound to provide a reaction mixture; and d) incubating the reaction mixture so that the activated antigen masking compound covalently binds to the red cell surface. In one embodiment, the reaction solution comprises a buffer having a concentration ranging from approximately 50 to 350 mM, preferably 75-350 mM, preferably 100-200 mM, preferably approximately 150 mM. In one embodiment, the reaction solution comprises a buffer having a pH in the range of approximately 8-10, preferably 8.5-9.5, more preferably 8.5-9, more preferably approximately 9. In another embodiment, the reaction solution comprises a buffer having a pKa of approximately 8.5-9.5, preferably approximately 9. In a further embodiment, the buffer is selected from the group consisting of TAPS, AMPD, TABS, AMPSO, CAPSO, and CHES. In a preferred embodiment, the buffer is CHES. In another embodiment, the reaction solution comprises dextrose. In a further embodiment, the dextrose is at a concentration of 50-300 mM, preferably approximately 75-200 mM, preferably approximately 100 mM. In a further embodiment, the wash solution comprises L-carnitine, preferably at a concentration of 2-100 mM, preferably 2-10 mM, more preferably approximately 5 mM. In one embodiment, the reaction solution comprises 150 mM CHES, 100 mM dextrose and 5 mM L-carnitine. In one embodiment, the volume of reaction solution used in step b is greater than or equal to the volume of the red cell concentrate. In one embodiment, the mixture of step c is at a hematocrit of 20-95%, also 30-95%, also 40-95%, also 50-95%, preferably 60-95%, or 60-80%. In one embodiment, the incubation is done at a temperature ranging from 4-40° C., preferably 20-25° C. In another embodiment, the incubation is for approximately 5-240 minutes, also 10-120 minutes, preferably 30-120 minutes, more preferably approximately 60 minutes. In one embodiment, the activated antigen masking compound has a molecular weight of approximately 2-40 kDa, in some embodiments 5-40 kDa, 10-40 kDa, 15-40 kDa, 20-40 kDa, 20-30 kDa, or 20-25 kDa. In a preferred embodiment, the activated antigen masking compound has a molecular weight of 5-40 kDa, preferably 15-40 kDa, preferably 20-40 kDa, more preferably 20-30 kDa. In one embodiment, the activated antigen masking compound is at a concentration of approximately 1-50 mM. For activated antigen masking compounds in the range of 15-40 kDa, the concentration used may be in the range of 1-30 mM, also 1-20 mM, also 2-15 mM or 2-10 mM. Antigen masking compounds in the range of 2-15 kDa can be used at higher concentrations, such as 5-50 mM, also 5-30 mM, 5-25 mM. In one embodiment, the activated antigen masking compound is in the range of 20-30 kDa at a concentration of 1-20 mM, also 2-10 mM. In a further embodiment, the activated antigen masking compound is a PEG derivative. In one embodiment, the activated antigen masking compound is an mPEG. In one embodiment the activated mPEG is selected from the group consisting of mPEG-SPA-NHS, mPEG-SBA-NHS, mPEG-SC, mPEG-βA-NHS, mPEG-6AC-NHS, mPEG-5AV-NHS, mPEG-4AB-NHS, mPEG-6AC-PFP, mPEG-6AC-TFE, mPEG-6AC-OEt, mPEG-6AC-PFT, mPEG-6AC-TFT, mPEG-6AC-4FTP, mPEG-6AC-TFET, and mPEG-6AC-SEt. In a preferred embodiment, the activated mPEG is selected from the group consisting of mPEG-6AC-NHS, mPEG-5AV-NHS, and mPEG-4AB-NHS. In a more preferred embodiment, the activated mPEG is mPEG-6AC-NHS.

In one embodiment, the present invention encompasses a method of preparing a red cell composition comprising a) providing i) a red cell concentrate, ii) an activated antigen masking compound, and iii) a reaction solution comprising a buffer at a concentration of 75-350 mM and a pH of 8-10; b) washing the red cell concentrate with the reaction solution to provide a washed red cell concentrate; c) mixing the washed red cell concentrate with the reaction solution and the activated antigen masking compound to provide a reaction mixture; and d) incubating the reaction mixture so that the activated antigen masking compound covalently binds to the red cell surface. In another embodiment, the reaction solution comprises a buffer having a pKa in the range of 8.5-9.5. In another embodiment, the reaction solution comprises a buffer at a concentration of 100-200 mM and a pH of 8-10, preferably 8.5-9.5.

In one embodiment, the present invention encompasses a method of preparing a red cell composition comprising a) providing i) a red cell concentrate, ii) an activated antigen masking compound, and iii) a reaction solution comprising a buffer at a concentration of 50-350 mM and a pH of 8.5-9.5; b) washing the red cell concentrate with the reaction solution to provide a washed red cell concentrate; c) mixing the washed red cell concentrate with the reaction solution and the activated antigen masking compound to provide a reaction mixture; and d) incubating the reaction mixture so that the activated antigen masking compound covalently binds to the red cell surface. In another embodiment, the reaction solution comprises a buffer having a pKa in the range of 8.5-9.5. In another embodiment, the reaction solution comprises a buffer at a concentration of 75-350 mM and a pH of 8.5-9.5.

In another embodiment, the above methods are performed using an automated system that provides the appropriate concentration of compound at the desired reaction hematocrit.

In one embodiment, the invention comprises a composition of modified red cells comprising red cells that have been prepared by the methods discussed above. In another embodiment, the invention comprises the use of such red cell compositions for transfusion using techniques known in the art. In one embodiment, the modified red cells have been washed following the reaction with antigen masking compound, preferably with a phosphate buffer or similar solution with a pH of approximately 7-8, preferably approximately pH 7. In another embodiment, after washing, the modified red cells are stored in a suitable storage solution, such as Adsol® (comprising 154 mM NaCl, 2.0 mM adenine, 41.2 mM mannitol, and 111.0 mM dextrose, Baxter Healthcare, Ill.), or Erythrosol™ (Erythrosol consists of 94 mL part A (25.0 mM sodium citrate, 16.0 mM disodium phosphate, 4.4 mM monosodium phosphate, 1.5 mM adenine, 39.9 mM mannitol), and 20 mL part B (8% dextrose), Baxter Healthcare, Ill.). In another embodiment, the post reaction wash solution is the same as the storage solution. Other red cell storage solutions include Nutricel® (70 mM NaCl, 2.2 mM adenine, 61 mM dextrose, 2 mM sodium citrate, 23 mM Na₂HPO₄, 2.2 mM citric acid, Miles, Ind.), Optisol® (150 mM NaCl, 2.2 mM adenine, 45.4 mM dextrose, 45.4 mM mannitol, Terumo) and SAGM (150 mM NaCl, 1.6 mM adenine, 50 mM dextrose, 29 mM mannitol).

II. Compounds for Quenching of Unreacted Antigen Masking Compound.

The present invention further contemplates the use of a quencher, which is intended to reduce or eliminate any unreacted antigen masking compound. Preferred quenchers would include a nucleophilic group capable of reacting with the coupling group of the antigen masking compounds. Examples of nucleophilic groups include, but are not limited to, thiol, thioacid, dithioic acid, thiocarbamate, dithiocarbamate, amine, phosphate, and thiophosphate groups. Additionally, the nucleophilic group could be an amino group, polyamino group, or a combination of thio and amino groups which could quench unreacted antigen masking compound. The quencher may be, or contain, a nitrogen heterocycle such as pyridine. The quencher can be a phosphate containing compound such as glucose-6-phosphate. The quencher also can be a thiol containing compound, including, but not limited to, glutathione, cysteine, N-acetylcysteine, mercaptoethanol, dimercaprol, mercaptan, mercaptoethanesulfonic acid and salts thereof (e.g. MESNA), homocysteine, aminoethane thiol, dimethylaminoethane thiol, dithiothreitol, and other thiol containing compounds.

Other thiol containing compounds include, but are not limited to, methyl thioglycolate, thiolactic acid, thiophenol, 2-mercaptopyridine, 3-mercapto-2-butanol, 2-mercaptobenzothiazole, thiosalicylic acid and thioctic acid. Exemplary aromatic thiol compounds include 2-mercaptobenzimidazolesulfonic acid, 2-mercapto-nicotinic acid, napthalenethiol, quinoline thiol, 4-nitro-thiophenol, and thiophenol. Other quenchers include, but are not limited to, nitrobenzylpyridine and inorganic nucleophiles such as selenide salts or organoselenides such as selenocysteine, thiosulfate, sulfite, sulfide, thiophosphate, pyrophosphate, hydrosulfide, and dithionite. The quenchers also can be in the form of a salt, such as sodium or hydrochloride salt. The quencher also can be a peptide compound containing a nucleophilic group. For example, the quencher may be a cysteine containing compound, for example, a dipeptide, such as GlyCys, or a tripeptide, such as glutathione or an amine containing compound such as polylysine. It is possible that the quencher may contain different nucleophilic groups, each of which are capable of quenching, such as the amine and thiol groups of glutathione. The quencher may also be immobilized on a solid support or be in a solid form.

III. Devices for the Reduction of Unwanted Compounds from the Red Blood Cell Composition.

The present invention further contemplates devices and methods for the reduction of unwanted compounds from the RBC composition. These compounds include unreacted antigen masking compounds, unwanted side products of these compounds, side products resulting from the reaction of these compounds during the washing process, side products resulting from the reaction of these compounds with quencher, excess quencher and quencher side products, and unwanted by-products of the processes. Removal devices contemplated for use in the present invention comprise an adsorbent material in a suitable matrix, which specifically and selectively reduces the concentration of unwanted compounds without significant effects on the in vitro or in vivo properties of the RBC composition. A thorough discussion of devices and methods for the reduction of compounds that could be applied to some of the compounds of the present invention can be found in PCT publication WO 98/30327, hereby incorporated by reference.

IV. Red Blood Cell Compositions Having Reduced Immunogenicity and Methods of Their Preparation.

The compositions contemplated by the present invention include an RBC composition that has been treated to substantially mask antigens so as to have significantly reduced immunogenicity when transfused to a recipient. In another embodiment, the composition of RBC has been reacted with an antigen masking compound such that the RBC antigens are substantially masked such that the transfusion of the treated RBC into an antigen mismatched animal would result in a reduced immune reaction compared to the immune reaction of the transfusion of an untreated RBC composition, wherein the treated RBC composition is suitable for in vivo use. A further embodiment contemplates that the function of the RBC composition is not significantly reduced from that of a comparable untreated RBC composition. In particular, the composition is suitable for in vivo use in that the in vivo function is not lowered significantly relative to an untreated composition. An additional embodiment of the present invention is a medicament comprising RBC wherein the RBC have been treated such that RBC antigens are substantially masked so that the transfusion of the treated RBC into an antigen mismatched animal would result in a reduced immune reaction compared to the immune reaction of transfusion of an untreated RBC composition.

Parameters for suitability are known to those of skill in the art and include, but are not limited to, in vivo survival and certain in vitro parameters useful in assessing RBC function. For example, it is desirable that the function of the treated RBC composition is such that the in vivo survival of the RBC after circulating 24 hours post transfusion is greater than approximately 40%, more preferably 50%, and more preferably 75%, in other embodiments, this survival rate of approximately 40%, more preferably 50%, and more preferably 70% is maintained 24 hours post transfusion of the treated RBC, which have been stored prior to transfusion for up to 7 days, 14 days, 21 days, 35 days, and 42 days at 4° C. In addition, certain in vitro parameters that are important in assessing the viability of the treated RBC composition include, but are not limited to, measurements indicating oxygen transport activity of the RBC (as measured by oxygen affinity), intracellular adenosine 5′-triphosphate (ATP) levels, intracellular 2,3-diphosphoglycerate (2,3-DPG) levels, extracellular potassium levels, reduced glutathione (GSH) levels, hemolysis or vesiculation of the RBC, pH, hematocrit, free hemoglobin levels, osmotic fragility of the RBC, deformability of the RBC by ektacytometry, ion homeostasis (Na⁺, K⁺ and SO₄⁻ fluxes), active cation transport (ouabain sensitive Na⁺ transport, bemetanide sensitive Na⁺, Na⁺ transport), glucose consumption and lactate production.

Methods for determining ATP, 2,3-DPG, glucose, hemoglobin, hemolysis, glutathione and potassium are available in the art. See for example, Davey et al., Transfusion, 32:525-528 (1992), the disclosure of which is incorporated herein by reference. Methods for determining RBC function are also described in Greenwalt et al., Vox Sang, 58:94-99 (1990); Hogman et al., Vox Sang, 65:271-278 (1993); Beutler et al., Blood, Vol. 59 (1982); and Beutler, Red blood cell Metabolism, 3rd edition, Grune & Stratton, (1984) the disclosures of which are incorporated herein by reference. Extracellular sodium and potassium levels may be measured using a Ciba Corning Model 614 K⁺/Na⁺ Analyzer (Ciba Corning Diagnostics Corp., Medford, Mass.). The pH can be measured using a Ciba Corning Model 238 Blood Gas Analyzer (Ciba Corning Diagnostics Corp.).

These measurements are compared to an untreated control RBC composition to determine whether the function of the treated composition has been significantly reduced. In one embodiment, an RBC composition having reduced immunogenicity will have extracellular potassium of no more than 3 times and more preferably no more than 2 times the level measured in an untreated control RBC composition 1 day after treatment. In another embodiment, hemolysis of the treated RBC composition is less than 5% after treatment and after up to 42 days storage at 4° C. In another embodiment, hemolysis of the treated RBC composition after storage at 4° C. is less than 3% after 28 days, more preferably less than 2% after 35 days, more preferably less than or equal to about 0.8% after 35 days, more preferably 42 days. In another embodiment, the treated RBC composition will have intracellular ATP levels that are within 75%, also 50%, more preferably 25%, and more preferably 10%, of the level of the untreated control composition directly after treatment, preferably within 50% after 28 days storage at 4° C., more preferably within 50% after 42 days storage at 4° C. In another embodiment, the treated RBC composition will have GSH levels that are within 75%, also 50%, more preferably 25%, and more preferably 10%, of the level of the untreated control composition directly after treatment, preferably within 50% after 28 days storage at 4° C., more preferably within 50% after 42 days storage at 4° C. In another embodiment, the treated RBC composition will have intracellular 2,3-DPG levels that are within 90%, more preferably 50%, and more preferably 25%, of the level of the untreated control composition directly after treatment, also preferably within 50% of the untreated control after 7 days storage at 4° C.

In another embodiment of the present invention, it is contemplated that the treated RBC have significantly reduced immunogenicity (i.e. reduced immune reaction) relative to an untreated RBC control. Certain in vitro assays, known to those skilled in the art, may be carried out to assess immunogenicity of a treated RBC relative to an untreated control. In vitro assays include, but are not limited to, ABO reactivity agglutination, measurement of RBC aggregation as a function of antibody added to the composition, reactivity to minor antigens, ELISA assay to measure direct binding of antibody, and analysis of bound fluorescent antibody to assess levels of modification by antigen masking compounds (e.g. Example 6).

As an example of ABO reactivity assessment, a composition containing treated RBC is reacted with serum containing a suitable antibody (e.g. treated type A RBC would be reacted with serum containing anti-A antibodies) and agglutination of the RBC is observed. The reaction is repeated with serially diluted aliquots of the antibody containing serum until no agglutination is observed. Standard blood typing assays used in blood banks involve such an agglutination assay, where typically one drop of antibody solution is mixed with one drop of the red cell solution and observed for agglutination of the red cells. This is typically done on a microscope slide for anti-A and anti-B antibodies. An embodiment of the present invention contemplates a treated RBC composition which requires at least a 2³ fold lesser dilution, 2⁴ fold lesser dilution, 2⁵ fold lesser dilution, 2⁶ fold dilution, or at least a 2⁷ fold lesser dilution of the antiserum relative to that required for an untreated control RBC composition in order to observe lack of agglutination. In a preferred embodiment, the red cells do not react with antibodies to any red cell antigens when tested with a standard agglutination test. Another example is an ELISA assay to measure the binding of antibodies to the RBC antigens using an anti-human IgG conjugated to alkaline phosphatase. Another embodiment of the present invention contemplates a treated RBC composition which is Rh positive (i.e. has the D antigen) in which binding of an anti-D antibody to the treated RBC relative to an untreated Rh positive RBC control, as measured by such an ELISA assay, is reduced by at least 75%, preferably at least 90%, preferably at least 95%, and most preferably more then 99%. The immunogenicity of minor antigens, including the D antigen, which are generally implicated in alloimmunization, can also be tested in vitro. The established system rates the reaction on a scale of 0-4+, 0 being no reaction, 4+ being the highest level of agglutination [Walker et al., AABB Technical Manual, 10th Ed., pp 528-537 (1990)]. These agglutination assays are typically done in a test tube and are scored by those skilled in the art. Minor antigens that have been implicated in alloimmunization include Jk^{a}, E, K, Bg, Lu^{a}, P₁, D, Sd^{a}, Fy^{a}, M, Yk^{a}, A₁, Le^{a}, Kp^{a}, C, e, and I [Heddle et al., Brit. J. Hemat. 91:1000-5 (1995)]. Another embodiment of the present invention contemplates a composition containing treated RBC which shows in vitro reactivity in this assay for anti-D, anti-Jk^{a}, anti-E, anti-C, anti-e, or anti-K of less than or equal to 2+, preferably less than or equal to 1+, most preferably 0 on this rating scale of 0-4+. In one embodiment, the treated RBC that show no reaction with any minor antigens may have some reactivity with the A or B antigens. Such compositions would be useful for alloimmunized subjects and would have to be crossmatched for the A and B antigens.

A gel card system using A/B/D Monoclonal Grouping Card™ kit (Micro Typing Systems, Pompano Beach, Fla.) can be used to look at the reactivity with A, B, and D antibodies (see Example 14). The gel cards contain either A, B, or D antibodies within the gel such that when a red cell is passed through the gel by centrifugation, it will agglutinate with the antibody if it contains an antigen that can bind the antibody. The agglutinated red cells will remain at the top of the gel while intact red cells pass through to the bottom of the gel, and the cell type can be easily determined by which antibody causes the agglutination. The resulting gels can be assigned a number of either 0, 1+, 2+, 3+, or 4+, where 0 indicates essentially completely intact cells (i.e. no reactions) and 4+ indicates complete agglutination. The effect of antigen masking on the antigens can be readily assessed by comparing the gel cards for a modified red cell compared to an unmodified red cell. Ideally, a red cell composition that shows agglutination with a particular antibody will show no reaction with the same antibody after it has been reacted with an antigen masking compound.

In one embodiment of the present invention, the modified RBC result in reduced binding of a fluorescent anti-type antibody as observed by FACScan™ (Becton, Dickinson and Co., N.J.). An analysis. In some embodiments, a modified red cell is considered to be adequately antigen masked if the anti-type antibody binding is less than 30%, also less than 20%, preferably less than 10%, more preferably less than 5% of the binding to the same source of red cells that has not been unmodified. In one embodiment, there is essentially no binding of the anti-type antibody to the modified red cells. Essentially not binding is observed when the FACScan peak for the modified red cells is less than or equal to the peak observed for a negative control red cell (i.e. a red cell that is not reacted with the fluorescent anti-type antibody).

In one embodiment of the present invention, the treated RBC result in a reduced immune response such that infusion into an ABO mismatched recipient (e.g. treated donor type A RBC infused into a type B recipient) would not result in an acute hemolytic reaction. In another embodiment, infusion of treated donor RBC into a recipient who is allosensitized (i.e. has developed antibodies to a minor antigen present on the untreated donor RBC, or an antigen resulting from pathogen inactivation treatment) would not result in an immune reaction to the alloantigens and rapid clearance of the RBC. It is also possible to use certain in vivo assays to assess the immune response to a treated RBC relative to an untreated control. In vivo survival studies may be done to assess the immune response, for example by assaying in vivo survival of treated sheep RBC in mice, or preferably in vivo survival in a model species, such as canines, of transfused RBC wherein the untreated RBC would elicit an immune response (i.e. antigen mismatched RBC). An embodiment of the present invention is one in which the in vivo survival of treated dog RBC is substantially increased over that of an untreated dog RBC from a donor which is antigen mismatched to the recipient. In general, substantially increased survival would be a survival of an RBC treated to reduce an immune response of approximately 2×, more preferably 5× and more preferably 10× the survival of an untreated RBC control. Such an increase in the survival is most likely the result of the reduction in the immunogenicity of the treated red cell due to the masking of the red cell antigens.

The present invention also contemplates methods of use comprising the above mentioned compositions and medicaments. An example of a method of use comprises the delivery of the composition or medicament into an individual in need of an RBC transfusion. Another embodiment contemplates an RBC processing system comprising compositions or medicaments as described above and a suitable container for storing the RBC composition wherein the RBC composition is suitable for delivery to an individual. In a preferred embodiment, the container is a blood bag.

The present invention further contemplates methods of producing an RBC composition that has significantly reduced immunogenicity. In a preferred embodiment, prior to adding the non-immunogenic compound that covalently binds to RBC, the red cells are washed with a suitable buffer such that the extracellular pH is optimal for reaction of the non-immunogenic compound with the RBC. A preferred buffer is one that results in an extracellular pH of approximately 8-9. Preferred buffers are buffers having an adequate buffering capacity at a pH range of 8-10, preferably 8.5-9.5, preferably 8.5-9, or approximately pH 9. Preferred buffers will comprise a buffer at a concentration ranging from approximately 50 mM to 350 mM, preferably 75 mM to 350 mM, preferably 100 mM to 200 mM, preferably approximately 150 mM at a pH of 8-10, preferably 8.5-9.5, preferably 8.5-9, or approximately 9.

In another embodiment, the method comprises adding a compound having a PEG group attached to a coupling group to an RBC composition, to create a mixture, and incubating said mixture to create an incubated mixture under conditions wherein said PEG group is covalently attached to the RBC to a level in which any RBC antigens are substantially masked from recognition by an allogeneic immune system resulting in an RBC composition that has significantly reduced immunogenicity, remains functional, and is suitable for in vivo use. In one embodiment of this method, a portion of the coupling group remains between the PEG group and the RBC while in another embodiment, the coupling group is eliminated, resulting in direct attachment of the polyethylene group to the RBC. In another embodiment, the treated RBC composition is additionally washed or treated to reduce the level of any unreacted PEG compound or any unwanted side products of these reagents, or unwanted by-products of the process.

V. Selecting Compounds and Methods for Preparation of Non-Immunogenic Red Blood Cells.

In order to evaluate compounds and methods to decide if they would be useful in the present invention, two important properties should be considered: the compound and method's effect on the immunogenicity of the red blood cell, and the effect of the compounds and methods on the functioning of the red blood cell composition for its intended use. Screening techniques to measure these parameters are known to those of skill in the art.

Screening techniques for immunogenicity of cells include those utilizing antibodies to recognize cell surface antigens. For example, a screening technique used to evaluate the compounds of the present invention for their ability to reduce the immunogenicity of the red blood cells is to perform separate agglutination tests using anti-A, anti-B, and anti-D antibodies; an assay that determines the immunogenicity of the red blood cells. A screen of this type is described in detail in Example 1. The particular compounds and methods of the present invention can be optimized for the maximum reduction in the immunogenicity of the A, B, and D antigens using this assay. Those compounds and methods which result in at least a 2³ fold reduction in the dilution of the anti-A or anti-B antiserum needed to see no agglutination relative to the dilution needed for an untreated control RBC sample and those compounds and methods which result in a score of 1+ or lower for anti-D antiserum are expected to be reasonable candidates for preparation of RBC compositions of the present invention.

Additional screening techniques may utilize a detection label such as radioactive or fluorescent labeled compounds. In such assays, the amount of suitably labeled PEG on the surface of the RBC can be measured directly by isolation and analysis of the RBC membranes (ghosts) or other methods of partitioning measurement for the RBC. Alternatively, the amount of fluorescently labeled PEG (FPEG) on the surface of the RBC can be directly measured using a flow cytometer. The fluorescent signal can be correlated to the relative amount of fluorescent PEG used and compared to a standard curve using, for example, beads containing known amounts of fluorescently labeled molecules. Alternatively, the FPEG results can be cross validated to another quantitative assay. An example of the measurement of the modification density of PEG modified RBC is given in Example 7. Another method of measuring the modification density involves the use of a PEG that incorporates an unnatural amino acid into the coupling group of the compound. For example, the reactive coupling group could contain 6 amino caproic acid such that this group gets attached to the red cell. The modified red cell ghosts can be treated by dissolving in acid to hydrolize proteins to free the 6 amino caproic acid group, which can be quantified by HPLC. The number of 6 amino caproic acid groups per red cell can then be calculated, thereby generating the number of mPEG molecules on the RBC surface. Methods of the present invention will result in a preferred level of antigen masking compound bound per red cell of at least approximately 10⁴, also at least 10⁵, preferably at least 10⁶ or preferably 10⁶-10⁸ molecules of antigen masking compound per red cell. Higher levels of modification are preferred for complete antigen masking while lower levels are adequate for the reduction in viscosity at low shear rates.

Screening techniques used to evaluate RBC compositions and methods of the present invention include, but are not limited to, measurement of intracellular ATP, intracellular 2,3-DPG, extracellular potassium, hemolysis, osmotic fragility and oxygen transport activity as described in the examples below. Those compounds and methods, when assayed relative to an untreated control sample, which do not vary significantly from the control sample (i.e. are within an acceptable range according to current standards of blood banking practice) are expected to be reasonable candidates for preparation of RBC compositions of the present invention.

### EXPERIMENTAL

### EXAMPLE 1

### Determination of Aglutination Reaction of RBC of the Present Invention with Anti-A, Anti-B, and Anti-D Antisera

The process of antigen masking of red cells is carried out under appropriate conditions on CPDA-1 collected RBC. PEG derivatives are commercially available (e.g. Shearwater Polymers Huntsville, Ala.). Agglutination reactions of the treated RBC are assayed by standard techniques as described in Walker et al., AABB Technical Manual, 10th Ed., pp. 528-537 (1990). The agglutination reaction is assessed on serially diluted samples. The dilution level at which agglutination no longer is observed is recorded for treated RBC compared to untreated RBC. This assay is carried out using type A RBC and anti-A antibody or type B RBC and anti-B antibody. The processing of the RBC with respect to antigen masking can be optimized in part based on this assay.

Similar assays can be done using Rh positive RBC and anti-D antiserum. In this assay, the agglutination will be scored as described in the AABB technical manual. The treated RBC will be compared to an untreated control sample to assess ability of the process to mask the D antigen.

### EXAMPLE 2

### Assessment in vitro of RBC Function After Processing of RBC

The intracellular adenosine-5′-triphosphate (ATP), intracellular 2,3-diphosphoglyceric acid (2,3-DPG), extracellular potassium, extracellular and intracellular pH, and hemolysis levels are readily assessed following processing of the RBC with compounds and methods of the present invention. The results are compared to untreated control samples to assess whether the treated RBC are suitable for their intended use, such as transfusion. Intracellular ATP and 2,3-DPG are measured using a Sigma ATP Kit or 2,3-DPG kit respectively (Sigma, St. Louis, Mo.). The ATP kit was used following Sigma procedure No. 366-UV hereby incorporated by reference. Extracellular potassium levels can be measured using a Ciba Corning 614 K⁺/Na⁺ Analyzer (Ciba Corning Diagnostics Corp., Medfield, Mass.). The extracellular pH can be measured by centrifuging the cells at 4° C. for 15 minutes at 12,000×g and removing the supernatant. The supernatant pH is measured on a standard pH meter at room temperature (e.g. Beckman, Epoxy Calomel electrode). For the intracellular pH, the remaining pellet in a centrifuge tube is capped and stored at approximately −80° C. for at least 2 hours, then lysed by adding deionized water. The lysed sample is well mixed and the pH of the solution is measured either at room temperature using a standard pH meter or at 37° C. using a Ciba-Corning model 238 blood gas analyzer.

### EXAMPLE 3

### Evaluation of the Oxygen Affinity of the Processed RBC

Following the processing of RBC with the compounds and methods of the present invention, oxygen affinity of the RBC samples is measured with a Hemox analyzer. The Hemox analyzer is pre-equilibrated at 37° C. Fifty μL of the RBC sample is mixed with 3.97 mL Hemox buffer solution (TCS Scientific Corp., New Hope, Pa.), containing 20 μL of 20% Bovine Serum Albumin (TCS Scientific Corp.) and 10 μL anti-foaming reagent (TCS Scientific Corp.) before transferring into the Hemox Analyzer cuvette. After the diluted sample is drawn into the cuvette, the temperature of the mixture is equilibrated with stirring for 8 minutes at 37° C. Subsequently, the diluted sample is fully oxygenated by exposure to air for 8 minutes. The instrument is calibrated for the partial pressure reading and the degree of hemoglobin saturation for each sample. The log ratio of the solution absorption at 560 to the absorption at 570 nm is recorded on the Y-axis while the partial pressure of oxygen (pO₂) obtained from a Clark electrode is recorded on the X-axis. The X-axis is calibrated by assigning values of 0 and the maximum calculated pO₂ for the day to readings obtained from 100% nitrogen and 100% air. The Y-axis is calibrated by assigning values of 0 and 1 to readings obtained from hemoglobin equilibrated under nitrogen or oxygen, respectively. For each sample an oxygen affinity curve is obtained by lowering the pO₂ through the introduction of nitrogen to the space above the liquid sample and measuring the percent of oxygen saturation of hemoglobin. The numerical data is converted to a graph of the oxygen affinity curve through the use of the computer program Kaleidagraph 3.0.5 (Synergy Software, Reading, Pa.) and the P₅₀ is determined from the half point of the curve. Measurements can be made on treated samples and compared to measurements of untreated control samples.

### EXAMPLE 4

### Evaluation of the Osmotic Fragility of the Processed RBC

The osmotic fragility of samples is measured for RBC processed with compounds and methods of the present invention and compared to untreated control samples. Reagent is prepared at 0.1, 0.2, 0.3, 0.35, 0.4, 0.45, 0.5, 0.55, 0.6, 0.65, 0.75, and 0.9% PBS (1.0% PBS is 9 g NaCl, 1.365 g Na₂HPO₄, and 0.186 g NaH₂PO₄ to a volume of 1 liter in water). A 10 μL aliquot of RBC sample is added to 1.0 mL of each of these solutions, mixed gently and incubated at room temperature for 30 minutes. After incubation, the sample is mixed gently and centrifuged for 2 minutes at 2,000×g. A spectrophotometer is zeroed with water and the absorption of the supernatant of the sample is measured at 540 nm. The % lysis is calculated using the following formula, in which the 0.9% PBS sample is considered background lysis and the 0.1% PBS sample is considered to be 100% lysis.

% lysis=(A₅₄₀−0.9% A₅₄₀)÷(0.1% A₅₄₀%−0.9% A₅₄₀)×100

The % lysis is plotted as a function of the %PBS and the plots are compared for treated RBC and untreated control RBC.

### EXAMPLE 5

### Measurement of Hemolysis of a PEG Modified Red Cell

The hemolysis of a red cell composition can be assessed by comparing the hemoglobin concentration in the supernatant as compared to a sample that is 100% lysed. The hematocrit of a red cell composition is measured using a hematocrit centrifuge and reader. To prepare a test sample, the red cell composition is centrifuged at 12000×g for 2 minutes, and the supernatant removed. The centrifuge step is repeated on the supernatant and 100 μl of the final supernatant is diluted into 1 mL of Drabkin's reagent (Sigma). A 100% lysis sample is prepared as a standard by dilution of 5 μl of the red cell composition into 1 mL of Drabkin's reagent. The absorbance of the test and standard samples is measured at 540 nm and the hemoglobin(Hb) concentration is measured as Hb(g/dL)=A₅₄₀×dilution factor/6.85. From this, the percent hemolysis is calculated as (100-hematocrit)×Test Hb (g/dL)/Standard Hb (g/dL). This can be measured at various steps throughout the processing of the red cells to assess the effects of additives on the hemolysis of the red cells.

### EXAMPLE 6

### Flow Cytometry Analysis of RBC to Assess Levels of PEG Modification

A unit of ABO-typed whole blood (Sacramento Blood Center, Calif.) is leukofiltered according to standard blood banking methods. The RBC are washed with a buffer comprising 150 mM CHES at a pH of 9.0 to eliminate plasma proteins and adjust the pH of the extracellular domain to the desired value for the reaction. A solution of activated mPEG (5 kDa) is prepared in the CHES buffer and an aliquot of the RBC suspension is added to this solution resulting in a final concentration of mPEG of 22 mM in the extracellular volume at a hematocrit of 40%. The solution is mixed by gentle vortexing and inversion and incubated for 1 hour at room temperature. Following this room temperature incubation, the solution is washed three times with blood bank saline (BBS, 154 mM NaCl, Baxter Healthcare) to remove any excess mPEG and any other reaction side products. Following this wash, Adsol is added to a final hematocrit of 40%. The resulting RBC suspension is stored at 4° C.

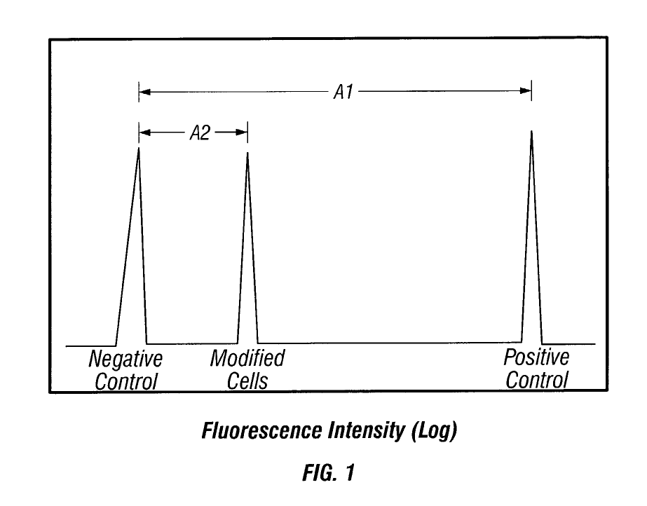

The modified cells are analyzed for their ability to bind fluorescently labeled antibody with a flow cytometry method using a FACScan. An aliquot of cells is centrifuged and the supernatant removed. A 50 μL portion of RBC (approximately 1×10⁶ cells) is incubated at room temperature for 1 hour with 5 μL of an appropriate stock antibody solution (i.e. antibody would bind non mPEG modified RBC, e.g. anti-A FITC conjugate BRIC-145, anti-B FITC conjugate BGRL1, or anti-D FITC conjugate BRAD-3 depending on the blood type, International Blood Group Reference Laboratory, UK). The cells are subsequently washed with BBS to remove the excess of the antibody and are analyzed by flow cytometry for bound fluorescent antibodies. The level of bound fluorescent antibodies is compared to either non MPEG modified cells (positive control) or cells which are not incubated with FITC antibody (negative control). The relative degree of PEG modification is estimated based on the ratio of the population maximum fluorescence (test article−negative control)/(positive control−negative control). This is represented as A2/A1 in FIG. 1. This calculation can be reported as a percent binding of antibody relative to a positive control.

### EXAMPLE 7

### Measurement of Modification Density for Pegylated RBC

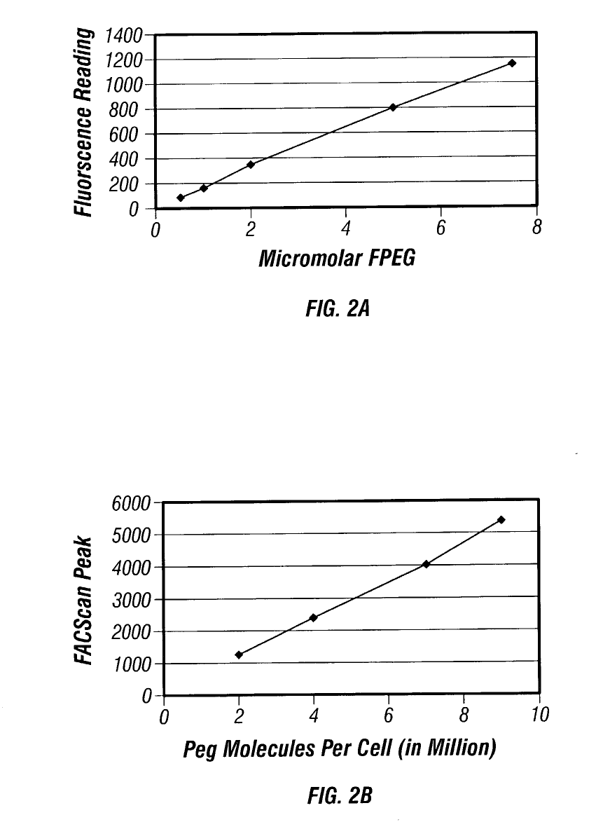

Leukofiltered RBC (approximately 60% hematocrit) containing a suitable additive solution such as Erythrosol are centrifuged to an 80-95% hematocrit (red cell concentrate), washed twice with pH 9.0 CHES buffer (150 mM CHES, 50 mM NaCl) and subsequently diluted to a hematocrit of 40% into a CHES buffer solution containing mPEG-SPA-NHS (or other activated PEG) at an appropriate concentration. In addition, the activated PEG is a mixture of the activated mPEG plus FITC labeled activated PEG (FPEG) with the same coupling group, which bears a fluorescent label on the end opposite the coupling group. Alternatively, the activated mPEG is modified with other detectable labels, such as a radioactive isotope. A 50:50 mixture of mPEG-SPA-NHS to FPEG-SPA-NHS is used for this experiment. The reaction is allowed to proceed for 2 hours at room temperature (RT) and the cells are subsequently washed to remove the reaction side products and any fluorescent label that is not attached to the red cells. RBC concentrate (200 μL) is subsequently used to make ghost membranes through controlled lysis with chilled hypotonic lysis buffer (1600 μL, 7.5 mM sodium phosphate, 1 mM NaEDTA, pH 7.5). The resulting ghosts are isolated through centrifugation (14000×g; 2 min) and washed a total of 4 times with chilled lysis buffer and then taken up in a 250 μL volume of the same buffer. SDS is added to the suspension to a final concentration of 1% SDS in order to achieve complete dissolution of the membranes. The resulting solution is further diluted 5 fold in lysis buffer and then analyzed for fluorescent label content (λ_{exc}=490 nm, λₑₘₘ=525 nm). The amount of fluorescent label is quantitated versus a standard curve prepared by adding specific amounts of FPEG in the dissolved ghost membranes in a lysis buffer containing SDS, prepared as per the reacted samples above. The fluorescence reading is plotted against the known concentration of FPEG added to the ghost membrane preparation and this curve is used to calculate the FPEG concentration corresponding to a fluorescence reading in the ghosts that have been reacted with the activated FPEG. Based on this calculated concentration of FPEG on the ghosts (or the concentration of another suitable label) and the number of cells used to prepare the ghosts, the amount of FPEG per cell (membrane modification density) can be calculated for a given experiment (FIG. 2A). From the FPEG:mPEG ratio, the number of total PEG molecules per cell can be calculated. An aliquot of the red cells that are modified with the FPEG can also be analyzed by FACScan (counting a set number of red cells). The calculated PEG molecules per cell can be plotted against the FACScan peak value (FL1-Height) and this curve can be used on new samples to calculate the amount of binding directly from the FACScan reading of the modified red cells (FIG. 2B).

As a an example of the calculations involved, ghosts were prepared at a level of 1.3×10⁹ cells and dosed with known concentrations of FPEG and the fluorescence measured to generate the line from FIG. 2A. Ghosts were prepared from 5 kDa mPEG-SPA-NHS/FPEG-SPA-NHS modified red cells and the modification density was measured at each concentration of PEG used in the reaction. The 13.2 mM PEG sample gave a fluorescence reading of 1495, which from FIG. 2A was calculated to be 9.8 μM. As the actual samples were only 50% FPEG, the total PEG for this sample was 19.6 μM. Based on the known cell number in the sample (1 mL volume), the total number of moles of PEG per cell was calculated as 19.6 μM×10⁻⁶ (mole/μmole)×10⁻³ (L/mL). The moles of PEG per cell was then 1.96×10⁻⁸ moles/1.3×10⁹ cells. Using Avogadros number, the total amount of PEG per cell is calculated to be 9×10⁶.

An additional method of use of the FPEG approach for the measurement of RBC PEG modification is achieved through the use of flow cytometry analysis of the pegylated RBC using a FACScan device. The RBC are directly analyzed for fluorescence intensity through a commercial device. The number of PEG molecules attached to the RBC surface is proportional to the percent of active FPEG in the active PEG. The FACScan fluorescent signal intensity is proportional to the PEG content. A standard curve of PEG modification done either through the method above or by comparison to beads containing known amounts of fluorescent molecules on them can be used to quantify fluorescent label amounts. Beads used in a FACScan device are commercially available and can be prepared to custom specifications (Bangs Laboratories, Fishers, Ind.).

An alternative method for the quantitation of the PEG molecules is the use of radioactively labeled activated mPEG (labeled with covalently attached ³H, ¹⁴C or other appropriate radioactive atom). The RBC are washed after the end of the PEG modification procedure and then the washed RBC are lysed, decolorized and the radioactivity content is measured through liquid scintillation. The extent of PEG modification is calculated using the specific activity of the radiolabeled activated mPEG.

Another method involves the use of an mPEG that contains an unnatural amino acid in the coupling group, such as mPEG-6AC-NHS (6-amino caproic acid is an unnatural amino acid). The reaction of this with red cells will deposit a number of the unnatural amino acids on the surface of the red cells that corresponds to the number of mPEG molecules on the surface of the red cells. The RBC are lysed after PEG modification along with control RBC that are unmodified. Ghosts are prepared from both populations for a known number of cells, the samples are treated to release free amino acids, and the amino acid content is measured for both preparations through the use of an HPLC assay (Sartore et al., Applied Biochemistry and Biotechnology 31:213, 1991). For example, with mPEG-6AC-NHS, the 6AC content will be compared to the number of natural amino acids. Since the control sample will give you the number of natural amino acids per red cell, the ratio between 6AC and the natural amino acids can be used to quantify the amount of 6AC per red cell, which gives the amount of mPEG per red cell. For a 5 mM 20 kDa mPEG-6AC-NHS treatment of RBC at a hematocrit of 40%, an amount of 6 AC equal to 2×10⁶ molecules per red cell was measured. Alternatively, the number of 6AC can be determined for a known number of red cells and the mPEG per red cell can be calculated directly.

The modification density for an antigen masking compound of a certain size can be determined as a function of the concentration used and correlated with agglutination assays or antibody binding assays to estimate the level of modification density necessary to get adequate coverage of the red cell antigens.

### EXAMPLE 8

### The Effect of Buffer Washes on the pH and Extent of PEG Modification of RBC

A unit of A+ whole blood (Sacramento Blood Center, CA) was leukofiltered according to standard blood banking methods. The RBC were centrifuged at 4° C. at 4100×g for 6 minutes and the plasma was removed to give a red cell concentrate. The cells were then aliquoted into three different tubes.

The effect of washing on the extracellular pH and the amount of PEG modification was studied by either washing four times with a buffer volume 14× the volume of RBC, washing two times with 1× the volume of RBC or not washing the RBC with any buffer.

In the first treatment, the RBC (2 mL, approximately 95% hematocrit) were washed four times with 14× volume of pH 8.0 HEPES buffer (150 mM HEPES, 50 mM NaCl). mPEG-SPA-NHS (5 kDa, 221 mg) was weighed and dissolved completely in the HEPES buffer (1.3 mL) by vigorous vortexing. The dissolved mPEG-SPA-NHS was immediately added to the washed RBC to a hematocrit of 60% and mixed, resulting in an mPEG-SPA-NHS concentration 13.6 mM (this is the total concentration. The effective concentration is in the extracellular volume only, which is approximately 34 mM). A control reaction (no PEG) was also run in a separate container.

In the second treatment, the RBC (7.5 mL, approximately 95% hematocrit) were washed two times with a volume of HEPES buffer equal to the RBC volume. MPEG-SPA-NHS (5 kDa, 825 mg) was weighed and dissolved completely in HEPES buffer (7.5 mL) by vigorous vortexing giving a 22 mM solution of mPEG-SPA-NHS (concentration in the extracellular volume). The dissolved mPEG-SPA-NHS was immediately added to the washed RBC and mixed (hematocrit of approximately 48%). A control reaction (no PEG) was also run in a separate container.

In tube three, unwashed RBC (7.5 mL) were used. mPEG-SPA-NHS (5 kDa, 825 mg) was weighed and dissolved completely in HEPES buffer (7.5 mL) by vigorous vortexing giving a 22 mM solution of mPEG-SPA-NHS (concentration in the extracellular volume). The dissolved PEG was immediately added to the RBC and mixed (hematocrit of approximately 48%). A control reaction (no PEG) was also run in a separate tube.

The reaction was allowed to proceed for 1 hour at RT in each of the three tubes and the cells were subsequently washed with 2× volume of BBS to remove the reaction side products.

Aliquots were removed from each tube after each wash to determine the pH and extent of PEG modification (measured as described in Example 6). A standard calibrated pH meter (Beckman, Epoxy Calomel electrode) was used at room temperature for the extracellular pH measurements. For the intracellular pH, for values above pH 8, a 0.5 mL RBC sample in a 1.5 mL microfuge tube is prepared by centrifuging at 4° C. for 15 minutes at 12,000×g. The supernatant is removed and the tube capped immediately. The cells are frozen at approximately −80° C. for at least 2 hours. While still frozen, 1 mL of deionized water is added to the tube, the solution warmed to room temperature with mixing and the lysed sample is well mixed before reading the pH using the Epoxy Calomel electrode at room temperature. For values below pH 8, the lysed sample is analyzed on a Ciba-Corning Model 238 blood gas analyzer. which measures the pH at 37° C. Control experiments showed the presence of PEG did not affect the pH values measured. Extracellular pH Values are indicated in Table 1.

TABLE 1
Extracellular pH of red cells with varying washing conditions.

| | 14x volume wash | 1x volume wash | No wash |
|---|---|---|---|
| Treatment | Test & Control | Test & Control | Test & Control |
| Wash 0 | 7.18 | 7.18 | 7.18 |
| Wash 1 | 7.99 | 7.76 | — |
| Wash 2 | 8.00 | 7.87 | — |
| Wash 3 | 7.99 | — | — |
| Wash 4 | 7.98 | — | — |
| After Incubation | | | |
| 1 hr | | | |
| Test | 7.70 | 7.66 | 7.46 |
| Control | 7.93 | 7.86 | 7.66 |

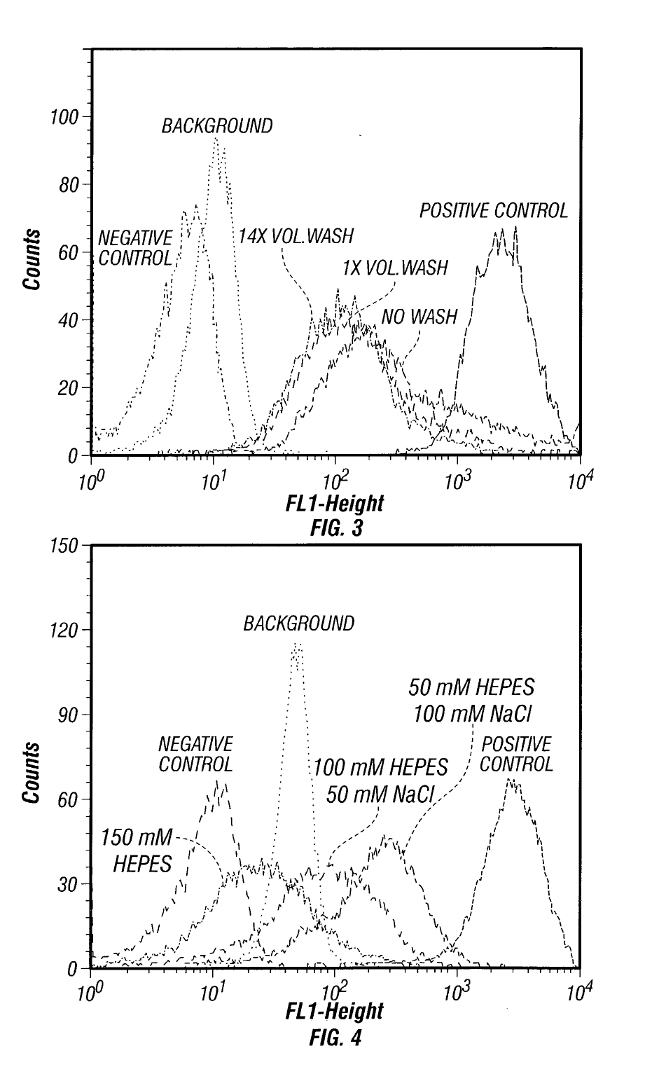

The pH of the RBC remained constant at approximately pH 8 when washed four times with 14× or two times with 1× buffer. The results indicate that a higher pH is maintained after the incubation with PEG (or without PEG control) in samples that incorporate the wash steps than in the sample with no wash step. It is also observed that the samples with PEG result in a lower pH than the controls, underlying the need for better pH control of the PEG process. The results of PEG modification on these samples showed, quantitatively, that PEG modification was similar when a high or a low washing volume was used (fluorescent anti-A antibody binding of 4.7%). Not washing the RBC however, resulted in a decrease in the rate and extent of PEG modification (7.8% anti-A antibody binding, FIG. 3).

### EXAMPLE 9

### The Effect of Buffer Strength on the Extent of PEG Modification of RBC

Packed RBC (B+) are prepared according to Example 8. The red cells were washed with three different isotonic buffers and reacted with 5 kDa MPEG-SPA-NHS in the wash buffer. The buffers used were isotonic, either 50 mM HEPES with 100 mM NaCl, 100 mM HEPES with 50 mM NaCl or 150 mM HEPES, all at pH 8.0. The red cells were washed four times with 14× volume of buffer. A solution of mPEG-SPA-NHS was prepared in each buffer and added to the blood to a hematocrit of 60%, resulting in a concentration of 5 kDa mPEG-SPA-NHS of 13.6 mM (approximately 34 mM in extracellular volume). The reaction was allowed to proceed for 1 hour at room temperature in each of the three buffers and the cells were subsequently washed twice with 2.5× volumes of blood bank saline (BBS) to remove the reaction side products. Aliquots were removed from each tube after each wash to determine the pH and the extent of PEG modification was measured as per Example 8. Extracellular (measured at room temperature) and intracellular (measured at 37° C.) pH Values are indicated in Table 2.

TABLE 2
Extracellular and intracellular pH values for red
cells reacted with varying buffer strength.

| | 50 mM HEPES | | 100 mM HEPES | | | |
|---|---|---|---|---|---|---|
| | 100 mM NaCl | | 50 mM NaCl | | 150 mM HEPES | |
| | Extra- | Intra- | Extra- | Intra- | Extra- | Intra- |
| Treatment | cellular | cellular | cellular | cellular | cellular | cellular |
| Wash 1 | 7.86 | 7.50 | 7.94 | 7.65 | 7.98 | 8.45 |
| Wash 2 | 7.91 | 7.52 | 8.04 | 7.89 | 8.04 | 8.80 |
| Wash 3 | 7.92 | 7.58 | 7.96 | 8.17 | 8.05 | 8.93 |
| Wash 4 | 8.06 | 7.56 | 7.96 | 7.96 | 7.90 | 8.86 |
| Incubation | 6.33 | 7.47 | 7.32 | 7.85 | 7.77 | 8.13 |
| BBS wash 1 | 6.86 | 7.44 | 7.51 | 7.78 | 8.00 | 7.86 |
| BBS wash 2 | 7.47 | 7.39 | 7.70 | 7.75 | 8.05 | 7.81 |

The results indicate that a higher pH is maintained during the incubation with PEG as the buffer strength is increased. However, this sample results in a higher intracellular pH that may be damaging to the red cells. The results of PEG modification on these samples showed, quantitatively, good correlation between a higher extracellular pH during incubation and an increase in the extent of PEG modification of the red cells (FIG. 4). The anti-B antibody binding showed improved PEG modification with increasing buffer concentration of 50, 100 or 150 mM HEPES (9.6%, 3.2% and 0.5% antibody binding, respectively). An experiment similar to this was done using 150 mM HEPES with 0, 50, or 100 mM NaCl on an A+ red cell unit to determine if the extracellular pH could be maintained with a more favorable intracellular pH. Extracellular and intracellular pH Values are indicated in Table 3.

TABLE 3
Extracellular and intracellular pH values for red cells
reacted with varying NaCl in 150 mM HEPES.

| | 150 mM HEPES | | 150 mM HEPES | | | |
|---|---|---|---|---|---|---|
| | 100 mM NaCl | | 50 mM NaCl | | 150 mM HEPES | |
| | Extra- | Intra- | Extra- | Intra- | Extra- | Intra- |
| Treatment | cellular | cellular | cellular | cellular | cellular | cellular |
| Wash 1 | 8.01 | 7.50 | 8.03 | 7.69 | 8.01 | 8.10 |
| Wash 2 | 8.02 | 7.62 | 8.03 | 7.67 | 8.05 | 8.38 |
| Wash 3 | 8.01 | 7.80 | 8.03 | 7.66 | 8.06 | 8.59 |
| Wash 4 | 8.01 | 7.67 | 8.03 | 7.86 | 8.05 | 8.28 |
| Incubation | 7.56 | 7.70 | 7.60 | 7.72 | 7.76 | 8.05 |
| BBS wash 1 | 7.61 | 7.62 | 7.62 | 7.57 | 8.01 | 7.49 |
| BBS wash 2 | 7.74 | 7.60 | 7.83 | 7.62 | 8.01 | 7.65 |

There was essentially no difference in the extent of PEG modification based on anti-A antibody binding, indicating that the favorable PEG modification conditions provided by the higher capacity buffer can be maintained while achieving improved intracellular pH by addition of sodium chloride. It is known that the red cells will take up the chloride ion and release hydroxide ion, thereby lowering the internal pH of the red cells.

### EXAMPLE 10

### The Effect of pH on the Extent of PEG Modification of RBC

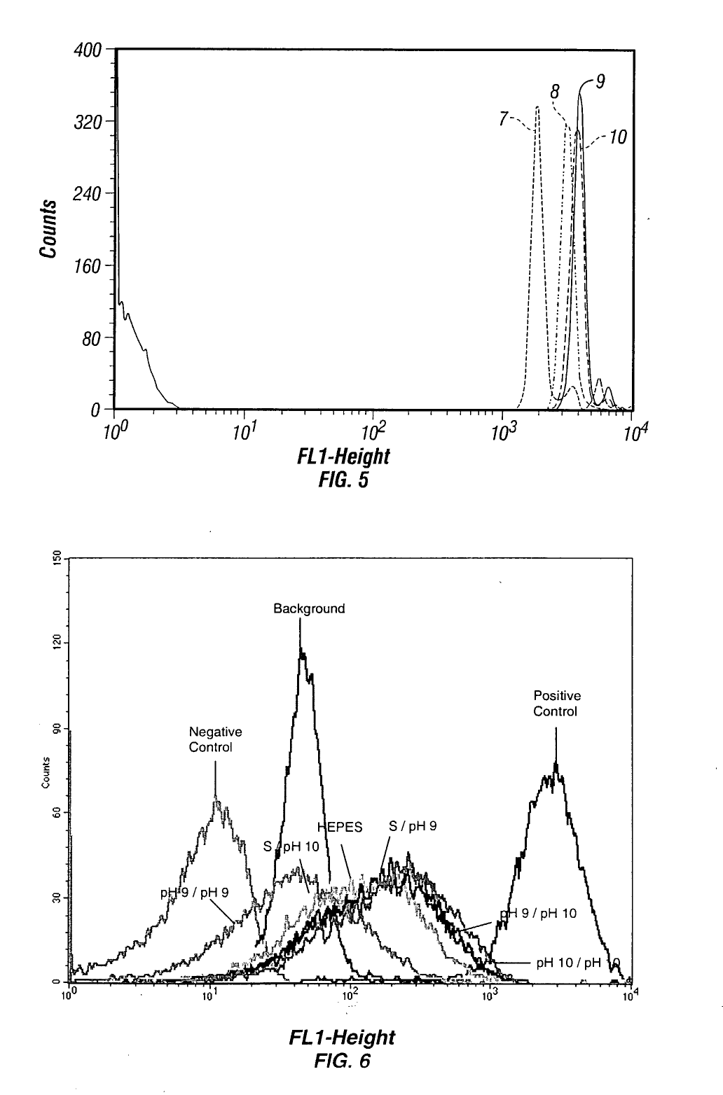

Red cell concentrates (B+) are prepared according to Example 8. The red cells were washed with four different buffers and reacted with a 50:50 mixture of 5 kDa mPEG-SPA-NHS and FPEG-SPA-NHS in the same buffer. The buffers used were PBS pH 7.0 (150 mM Na₂HPO₄, 50 mM NaCl), HEPES pH 8.0 (150 mM HEPES, 50 mM NaCl), CHES pH 9.0 (150 mM CHES, 50 mM NaCl), and CAPSO pH 10.0 (150 mM CHES, 50 mM NaCl). These particular buffers were selected to have good buffering capacity at the desired pH ranges. The red cell concentrates were washed twice with 1× volume of buffer. A solution of mPEG-SPA-NHS/FPEG-SPA-NHS was prepared in each buffer and added to the blood to give a final concentration of the PEG mixture of 13.2 mM at a hematocrit of 40% (approximately 22 mM in the extracellular volume). The reaction was allowed to proceed for 2 hours at room temperature in each of the four buffers. The pH was monitored at 30 minute intervals during the reaction and the amount of PEG bound to the red cells was assessed by measurement of the fluorescence associated with the red cells by FACScan analysis (FIG. 5). The fluorescent intensity peak value for the samples were pH 9=4032, pH 10=3786, pH 8=3220 and pH 7=1928. The results indicate that a buffer pH in the range of 8-10 is preferable with the pH 9 buffer giving the greatest amount of PEG modification of the cells. A similar experiment was done using the same buffers with the inclusion of 75 mM dextrose in all buffers. The PEG modification using the pH 10 buffer was reduced with dextrose while the others were not changed significantly. The fluorescent peak values for this study were pH 9=1155, pH 8=890, pH 10=784, pH 7=573. Note that the peak values are not comparable from one experiment to another as the extent of PEG modification depends on the red cells.

### EXAMPLE 11

### PEG Modification of Red Cells in CHES pH 9 Buffer, Effect of Pre Wash pH on the Extent of PEG Modification

Red cell concentrates (B+) are prepared according to Example 8. The red cell concentrates were washed twice with 1× volume of either BBS, CHES (150 mM CHES, 50 mM NaCl) pH 9.0, or CHES pH 10.0. A solution of mPEG-SPA-NHS was prepared in CHES pH 9.0 or 10.0 and added to the blood to give a final concentration of 5 kDa mPEG-SPA-NHS of 13.2 mM at a hematocrit of 40% (approximately 22 mM in the extracellular volume). The specific samples tested were as follows; a) BBS wash, pH 9.0 reaction, (b) pH 9.0 wash, pH 9.0 reaction, (c) BBS wash, pH 10.0 reaction, (d) pH 9.0 wash, pH 10.0 reaction, and (e) pH 9.0 wash, pH 10.0 reaction. A sample was also prepared using HEPES (150 mM HEPES, 50 mM NaCl, 75 mM dextrose) pH 8.0 for the wash and reaction buffer. The reaction was allowed to proceed for 2 hours at room temperature. The extent of PEG modification was measured as per Example 8 (FIG. 6, Table 4). Although the pre wash with saline may provide adequate masking of the antigens, the results indicate that washing with pH 9.0 is preferable to washing with saline prior to reacting using CHES pH 9.0 buffer.

TABLE 4
Anti-B antibody binding remaining after PEG modification with varying
wash and reaction conditions.

| Wash buffer | Reaction buffer | Percent antibody binding |
|---|---|---|
| CHES pH 9.0 | CHES pH 9.0 | 1.1 |
| BBS | CHES pH 10.0 | 3.7 |
| HEPES pH 8.0 | HEPES pH 8.0 | 5.1 |
| CHES pH 9.0 | CHES pH 10.0 | 6.9 |
| BBS | CHES pH 9.0 | 8.4 |
| CHES pH 10.0 | CHES pH 10.0 | 9.3 |

### EXAMPLE 12

### Reaction of a Full RBC Unit with 20 kDa mPEG-6AC-NHS

A unit of A+ whole blood was leukofiltered according to standard blood banking methods. The RBC, contained in a blood bag, were centrifuged at 4° C. for 6 minutes at 4100×g and the plasma was removed. The red cell concentrate (approximately 80% hematocrit) was washed with approximately an equal volume of 150 mM CHES pH 9, 100 mM dextrose, 5 mM L-carnitine (approximately 200 mL red cell concentrate and 200 mL buffer) and centrifuged as above, and the supernatant was removed. The wash procedure was repeated. A 10.7 g sample of 20 kDa mPEG-6AC-NHS was dissolved in 67 mL of 150 mM CHES pH 9, 100 mM dextrose, 5 mM L-carnitine and added to the approximately 200 mL washed red cell concentrate to give approximately 5 mM mPEG-6AC-NHS in the extracellular volume at a hematocrit of approximately 60%. The reaction mixture was gently mixed and incubated at room temperature for approximately 1 hour. Following incubation 200 mL of 150 mM Na₂HPO₄ pH 7, 100 mM dextrose, 5 mM L-camitine was added to the approximately 267 mL RBC sample. This was mixed and centrifuged at 4° C. for 6 minutes at 4100×g. The supernatant was removed and the red cell concentrate was washed again with 200 mL of the phosphate buffer. The wash buffer was removed and the final red cell concentrate was suspended in Erythrosol (Erythrosol is added as 94 mL part A and 20 mL part B(8% dextrose)), and stored at 4° C. The amount of anti-A antibody binding was assessed as per Example 6, agglutination tested as per Example 13 initially and after 21 and 42 days storage at 4° C. The anti-A antibody binding was 0% at all points and the gel cards showed no reaction as well. The hemolysis, ATP levels, potassium levels, and both intracellular and extracellular pH were measured. The potassium and pH measurements were taken at 37° C. These measurements were not taken initially but were taken after storage at 4° C. for 2, 7, 14, 21, and 42 days. These are compared to control samples, either red cells stored by standard methods (4° C. control prepared directly with Erythrosol) or red cells that are processed as above without the mPEG-6AC-NHS (wash control). The in vitro function results are found in Tables 5A-E. The results show that a full unit can be modified to adequately mask antigens and provide suitable values for hemolysis, potassium, ATP and both intracellular and extracellular pH.

TABLE 5A
Day 2 in vitro measurements of antibody binding and red cell function for
a full unit modified with 20 kDa mPEG-6AC-NHS.

| In vitro measurement | 4° C. control | Wash control | mPEG unit |
|---|---|---|---|
| % Hemolysis | 0.09 | 0.29 | 0.35 |
| ATP (μmol/g Hb) | 4.16 | 2.91 | 1.69 |
| Potassium (mM) | 7.35 | — | 24.3 |
| pHe (37° C.) | 6.89 | 7.7 | 7.49 |
| pHi (37° C.) | 6.87 | 7.38 | 7.39 |

— measurement not taken.

TABLE 5B
Day 7 in vitro measurements of antibody binding and red cell function for
a full unit modified with 20 kDa mPEG-6AC-NHS.

| In vitro measurement | 4° C. control | Wash control | mPEG unit |
|---|---|---|---|
| % Hemolysis | 0.10 | 0.27 | 0.47 |
| ATP (μmol/g Hb) | 4.21 | 3.24 | 2.37 |
| Potassium (mM) | — | — | — |
| pHe (37° C.) | 6.83 | 7.33 | 7.37 |
| pHi (37° C.) | 6.75 | 7.08 | 7.07 |

— measurement not taken.

TABLE 5C
Day 14 in vitro measurements of antibody binding and red cell function
for a full unit modified with 20 kDa mPEG-6AC-NHS.

| In vitro measurement | 4° C. control | Wash control | mPEG unit |
|---|---|---|---|
| % Hemolysis | 0.13 | 0.25 | 0.32 |
| ATP (μmol/g Hb) | 4.89 | 4.44 | 2.57 |
| Potassium (mM) | 30 | 37.9 | 57.2 |
| pHe (37° C.) | 6.74 | 6.96 | 6.88 |
| pHi (37° C.) | 6.64 | 6.76 | 7.14 |

TABLE 5D
Day 21 in vitro measurements of antibody binding and red cell function
for a full unit modified with 20 kDa mPEG-6AC-NHS.

| In vitro measurement | 4° C. control | Wash control | mPEG unit |
|---|---|---|---|
| % Hemolysis | 0.14 | 0.38 | 0.54 |
| ATP (μmol/g Hb) | 4.55 | 4.35 | 2.29 |
| Potassium (mM) | 35.9 | 40 | 55.5 |
| pHe (37° C.) | 6.66 | 6.78 | 7.02 |
| pHi (37° C.) | 6.61 | 6.60 | 6.76 |

TABLE 5E
Day 42 in vitro measurements of antibody binding and red cell function
for a full unit modified with 20 kDa mPEG-6AC-NHS.

| In vitro measurement | 4° C. control | Wash control | mPEG unit |
|---|---|---|---|
| % Hemolysis | 0.28 | 0.57 | 0.86 |
| ATP (μmol/g Hb) | 4.93 | 2.90 | 2.19 |
| Potassium (mM) | 38.0 | 38.9 | — |
| pHe (37° C.) | 6.50 | 6.30 | 6.70 |
| pHi (37° C.) | 6.39 | 6.24 | 6.47 |

### EXAMPLE 13

### Assay for Agglutination of Red Cells on Reaction with A, B, or D Antibodies

A non-immunogenic red cell composition can be assayed using an A/B/D Monoclonal Grouping Card™ kit (Micro Typing Systems, Pompano Beach, Fla.). The desired red cell sample at a hematocrit of approximately 40% is diluted to approximately 4% with MTS Diluent 2 Plus (typically, dilute 50 μl of red cells with 0.5 mL of diluent). A 10-12.5 μl aliquot of the red cell sample is added to an Anti-A/B/D microtube. Typically, six microtubes are prepared as a Gel Card and centrifuged using the MTS centrifuge. The Gel Card is then observed and scored for agglutination. Agglutination is graded as 0, 1+, 2+, 3+, or 4+. This range has 0 indicating no reaction with the red cells, all cells pellet at the bottom of the microtube and 4+ indicating complete agglutination with a layer of cells at the top of the gel). There may be cases where a mixed field results, i.e. there are some cells at both the top and bottom of the gel.

### EXAMPLE 14

### Measurement of FPEG Binding Using HEPES pH 8 vs. Triethanolamine at Various pH

Red cell concentrates (B+) were prepared according to Example 8. The red cell concentrates were washed twice with 1× volume of reaction buffer. The buffers used were HEPES pH 8.0 (150 mM HEPES, 50 mM NaCl, 75 mM dextrose) or triethanolamine(TE) pH 8.0, 8.5, or 9.0 (30 mM TE, 150 mM NaCl). The red cells are reacted with a 50:50 mixture of 5 kDa MPEG-SPA-NHS and FPEG-SPA-NHS. A solution of mPEG-SPA-NHS/FPEG-SPA-NHS was prepared in each buffer and added to the washed red cells to give a final concentration of the PEG mixture of 11 mM in the extracellular volume at a hematocrit of 40%. The reaction was allowed to proceed for 2 hours at room temperature in each of the four buffers. After the PEG reaction, all samples were washed with an equal volume of 150 mM Na₂HPO₄, 50 mM NaCl, 75 mM dextrose pH 7.0, centrifuged to red cell concentrate and washed again. The final red cell concentrate was suspended in an approximately equal volume of Adsol. The amount of PEG bound to the red cells was assessed by measurement of the fluorescence associated with the red cells by FACScan analysis. The results are found in Table 6. The results indicate that HEPES is preferred to TE buffer and that TE buffer is less effective as the pH is increased further from it's optimal buffering pH (TE pKa=7.9).

TABLE 6
Fluorescent PEG binding using HEPES vs TE buffers.

| Reaction buffer | Fluorescent peak value |
|---|---|
| HEPES pH 8.0 | 4255 |
| TE pH 8.0 | 3718 |
| TE pH 8.5 | 1685 |
| TE pH 9.0 | 835 |

### EXAMPLE 15

### PEG Modification of Red Cells at pH 7, 8, or 9

Red cell concentrates (B+) are prepared according to Example 8. The red cell concentrates were washed twice with an equal volume of reaction buffer. The buffers used were CHES pH 9.0 (150 mM CHES, 100 mM dextrose, 5 mM L-carnitine), HEPES pH 8.0 (150 mM HEPES, 100 mM dextrose, 5 mM L-carnitine) or phosphate pH 7.0 (150 mM Na₂HPO₄, 100 mM dextrose, 5 mM L-carnitine). A solution of 5 kDa mPEG-6AC-NHS was prepared in each buffer and added to the blood to give a final concentration of the mPEG-6AC-NHS of 22 mM in the extracellular volume at a hematocrit of 40%. The reaction was allowed to proceed for 1 hour at room temperature in each buffer. After the reaction, an equal volume of the pH 7.0 phosphate buffer was added to each sample, centrifuged and the supernatant removed to make red cell concentrate. This was washed with an equal volume of the phosphate buffer, centrifuged and the final red cell suspended in Erythrosol at approximately 60% hematocrit. The extent of PEG modification was assessed by measuring the binding of fluorescent anti-B antibody by FACScan. The hemolysis of the samples was also measured in the final storage solution as per Example 5. The results are found in Table 7. The pH 8.0 and 9.0 showed much better PEG modification than pH 7.0. While the extent of PEG modification was the same for the pH 8.0 and 9.0, the hemolysis was considerably less in the pH 9.0 buffer.

TABLE 7
Anti-B antibody binding and hemolysis results for red cells pegylated at
pH 7, 8, or 9.

| Reaction buffer | Percent antibody binding | Percent hemolysis |
|---|---|---|
| Phosphate pH 7.0 | 40.2 | 1.2 |
| HEPES pH 8.0 | 10.7 | 2.8 |
| CHES pH 9.0 | 10.7 | 1.5 |

### EXAMPLE 16

### Comparison of the Reduction of Immunogenicitiy for Red Cells Modified with 5 kDa vs. 20 kDa PEG

A unit of A+ whole blood was leukofiltered according to standard blood banking methods. The RBC were centrifuged at 4° C. at 4100×g for 6 minutes and the plasma was removed. The red cell concentrates were washed with an equal volume of CHES buffer, pH 9.0 (150 mM CHES, 50 mM NaCl), centrifuged as above, the supernatant was removed and the wash was repeated. An aliquot of the washed RBC (approximately 80% hematocrit) is diluted with an equal volume of the desired mPEG dissolved in CHES buffer. For 5 kDa mPEG-SPA-NHS, 99, 49.5, 24.75, and 12.375 mg were dissolved in 0.75 mL buffer and added to 0.75 mL RBC (approximately 40% hematocrit resulting in 22, 11, 5.5, and 2.75 mM mPEG, respectively, in the extracellular volume). For 20 kDa mPEG-SPA-NHS, 90, 45, 18, 9, and 4.5 mg were dissolved in 0.75 mL buffer and added to 0.75 mL RBC (resulting in 5, 2.5, 1, 0.5, and 0.25 mM mPEG, respectively, in the extracellular volume). The samples were gently mixed and incubated at room temperature for one hour. Following the incubation, 0.75 mL of PBS pH 7 is added, the samples are centrifuged at 4° C. at 4100×g for 6 minutes and the supernatant is discarded. This wash is repeated and the final red cell pellet is suspended in an approximately equal volume of Adsol. Each sample is assayed using the FACScan assay described in Example 6, using an anti-A FITC conjugate. The samples were also assessed for agglutination with anti-A or anti-D antibody using a gel card assay (Example 12). These results of percent antibody binding and agglutination are shown in Table 8.

TABLE 8
Anti-A antibody binding (FACScan) and anti-A and anti-D Gel card
scoring for 5 kDa vs 20 kDa PEG modified red cells.

| | | |
|---|---|---|
| mPEG-SPA-NHS | Percent Anti-A | Reaction Grade |

| Size/Concentration | Antibody Binding | Anti-A | Anti-D |
|---|---|---|---|
| No mPEG | 100 | 4+ | 4+ |
| 5 kDa/2.75 mM | 25.1 | 3+ | 2+ |
| 5 kDa/5.5 mM | 8.8 | 2+ | 0 |
| 5 kDa/11 mM | 2.6 | 1+ | 0 |
| 5 kDa/22 mM | 1.6 | 0 | 0 |
| 20 kDa/0.25 mM | 48 | 3+ | 4+ |
| 20 kDa/0.5 mM | 48 | 2+ | 2+ |
| 20 kDa/1.0 mM | 23.8 | 0 | 0 |
| 20 kDa/2.5 mM | 0.4 | 0 | 0 |
| 20 kDa/5.0 mM | 0 | 0 | 0 |

### EXAMPLE 17

### PEG Modification of Red Cells with 20 kDa mPEG at pH 8.5, 9.0, and 9.5

A unit of A+ blood was leukofiltered according to standard blood banking methods. The RBC were centrifuged at 4° C. at 4100×g for 6 minutes and the plasma was removed. Approximately 0.5 mL of the red cell concentrate were washed with 0.5 mL of CHES buffer, pH 8.0, 8.5, or 9.0 (150 mM CHES, 100 mM dextrose, 5 mM L-carnitine), centrifuged as above, the supernatant was removed and the wash was repeated. A 60 mg sample of 20 kDa mPEG-SPA-NHS was weighed out and dissolved in 0.5 mL of each buffer and added to the washed red cell concentrate to give approximately 40% hematocrit with 5 mM mPEG-SPA-NHS in the extracellular volume. This was inverted and gently vortexed to mix, then incubated at room temperature for about 1 hour. Following the incubation, samples were washed twice with phosphate buffer pH 7 (150 mM Na₂HPO₄, 100 mM dextrose, 5 mM L-carnitine) and resuspended in an approximately equal volume of Adsol. The anti-A antibody binding was measured by FACScan analysis and the samples were tested for agglutination using a gel card analysis (anti-A and anti-D). None of the red cells reacted with antibody in the gel card assay. For the FACScan analysis, the pH 8.5 and 9.0 samples showed essentially no binding while the pH 9.5 sample showed 1.9% antibody binding relative to the unmodified cells. While all three samples give adequate antigen masking, the pH 8.5 and 9.0 provided slightly better binding of the activated mPEG-SPA-NHS than pH 9.5.

### EXAMPLE 18

### Masking of Major and Minor Antigens with 20 kDa mPEGs

A unit of A+ blood was processed for modification with mPEG as per Example 16. The 20 kDa mPEG was used at 5 mM and was either mPEG-SPA-NHS (Shearwater) or mPEG-6AC-NHS (Example 19). In addition to analysis of anti-A antibody binding by FACScan and agglutination with gel cards, the reacted cells were sent to the SMF Center for Blood Research (Sacramento, Calif.) to be tested for cross reactivity to minor antigens. The antigens tested were A, A₁, B, D, C, E, c, e, K, k, Kp^{b}, Fy^{a}, Fy^{b}, Jk^{a}, Jk^{b}, S, s, Le^{a}, Le^{b}, P1, M, and N. Only A, A₁, D, C, c, e, k, Kp^{b}, Fy^{a}, Jk^{a}, S, Le^{b}, P1, and M showed reactivity with non PEG modified controls. For the samples modified with either 20 kDa mPEG, all but one antigen showed no reactivity. Antisera P1 showed a slight reaction (grade 1). Both mPEGs essentially completely blocked anti-A antibody binding by FACScan and showed no reaction on the gel cards for agglutination. This example demonstrates the ability to take a type A unit of red cells and make it equivalent to a type O unit, as well as negative to any antibodies to minor antigens.

### EXAMPLE 19

### Survival of mPEG Modified Human Red Cells Infused into Mice

In order to assess the immunogenicity of an mPEG modified red cell, it can be infused into another species and assessed for survival in a cross species model. Survival of the red cells suggests that it is not reactive with the immune system of the other species. In order to assess the survival, the red cells are labeled with PKH-26 (using PKH-26-GL kit from Sigma), a fluorescently labeled protein that tags the red cells so they can be measured by FACScan analysis. RBC are centrifuged for 2 minutes at 790×g. The packed red cells (2-3 mL) are resuspended with Diluent C (from Sigma kit) to about 10 mL. In another tube, 40 μl of PKH-26 dye solution is diluted into 10 mL of Diluent C. The two solutions are mixed by inverting in a 50 mL tube approximately 60 times in a minute. Inactivated fetal calf serum is added to a total volume of 50 mL, and this is centrifuged for 5 minutes at 790×g. The supernatant is removed and 50 mL of PBS (Ca and Mg free, Gibco) is added. The sample is centrifuged for 5 minutes at 790×g. This PBS wash is repeated for a total of four washes. The treated cells are transferred to a tube for FACScan analysis. The PKH-26 labeled red cells can then be mPEG modified and assessed for the effectiveness of the mPEG modification as described in the examples above. The PKH-26 labeled red cells (either mPEG modified or unmodified controls) can then be infused into mice through a tail vein injection. Blood samples can be removed over time and assayed by FACScan to see how many labeled red cells survive. Another possible method includes the reaction of the RBC with carboxyfluorescein diacetate succinimidyl ester (CFSE), which enters the cells and reacts with proteins in the cell. This causes the cells to be highly fluorescent without any membrane modification.

### EXAMPLE 20

### Modification of RBC Using Methods Known in the Art

Published methods for the attachment of activated PEG compounds were assessed. In one case, a 5 kDa activated PEG was used with a 30 mM triethanolamine (TE) buffer at pH8.6 with 150 mM NaCl [Blackall et al., Blood 97(2):551-556 (2001)]. The red cells were washed twice with 10 mM PBS pH 7 and the reaction was prepared at 9.6 mM combined PEG-SPA-NHS and FPEG-SPA-NHS at a hematocrit of 10% using the TE buffer. Alternatively, either PEG-SPA-NHS or cyanuric chloride PEG were used at 9.6 mM without the FPEG. The reaction was done for 1 hour at room temperature and the cells were washed twice with the PBS and suspended in PBS for analysis. This was compared to using a method of the invention where the red cells are washed twice with 150 mM CHES pH 9, 50 mM NaCl and reacted in the same buffer and washed with phosphate buffer pH 7 (150 mM Na₂HPO₄, 50 mM NaCl) using 11 mM 5 kDa combined mPEG-SPA-NHS and FPEG-SPA-NHS or 22 mM of the mPEG-SPA-NHS. The pH during the reaction step for the TE buffer protocol went from 8 down to 7.8 for the cyanuric chloride PEG and from 7.6 down to 7.5 for the SPA PEG. For the studies done without FPEG, the anti-A antibody binding was >98% for the TE protocol while it was approximately 9.6% for the CHES protocol. For the FPEG studies, the fluorescent measurement was 4255 for the CHES method and only 1459 for the TE method.

In another study, 5 kDa cyanuric chloride activated PEG (Sigma) was used at a 5 mM concentration in 50 mM K₂HPO₄, 105 mM NaCl pH 8.0 buffer at a 40% hematocrit [Bradley et al., Transfusion 41:1225 (2001)] to modify a type B+ red cell sample. This was prepared from whole blood without washing and was reacted for 30 minutes at room temperature and then washed three times with PBS (10 mM Na₂HPO₄, 105 mM NaCl pH 7.0). These were compared to 11 mM and 5.5 mM 10 kDa MPEG-SPA-NHS with CHES pH 9 as detailed above. The gel card agglutination showed no reaction with the CHES reacted samples while the phosphate buffered reaction showed some agglutination with anti-B (grade 2+) and a mixed field with anti-D antibody. The anti-B antibody binding by FACScan showed no binding with the 11 mM sample in CHES and 11.7% binding with the 5.5 mM sample in CHES while the phosphate buffered sample showed 63% binding of the antibody. These examples indicate considerable improvement in modification of the red cells with buffers of the invention compared to known methods.

### EXAMPLE 21

### Synthesis of mPEG-6AC-NHS, mPEG-5AV-NHS and mPEG-4AB-NHS Esters

N-6-[Methoxypoly(ethyleneglycol)]-oxo-aminocaproic acid 20 kDa (mPEG-6AC):

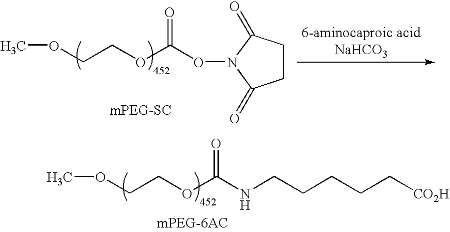

6-Aminocaproic acid (840 mg, 6.40 mmol) and NaHCO₃ (538 mg, 6.40 mmol) were dissolved in a mixture of H₂O (160 mL) and ethanol (50 mL). Methoxypoly(ethyleneglycol) succinimidyl carbonate (64.2 g, 3.20 mmol, prepared from the Methoxypoly(ethyleneglycol)-OH (Shearwater Polymers, Huntsville, Ala.) by known methods) was added and the mixture was stirred at room temperature for 3 h. HCl (1 N) was added until the solution reached pH 5. The resulting solution was extracted with CH₂Cl₂ (3×150 mL) and the combined organic extracts were dried (Na₂SO₄), filtered and concentrated under vacuum to give mPEG-6AC (60.11 g, 2.99 mmol, 93%) as a white solid.

¹H NMR (200 MHz, DMSO-d₆): δ_{H} 7.14-7.29 (m, 1 H), 4.07 (t, J=4.44, 2 H), 3.60-3.30 (m, 1806 H), 3.25 (s, 3 H), 2.89-2.99 (m, 2 H), 2.20 (t, J=7.32, 2 H), 1.10-1.60 (m, 6 H). IR; nujol, cm⁻¹, 1721, 1643, 1099.

N-6-[Methoxypoly(ethyleneglycol)]-oxo-aminocaproate N-hydroxysuccinimide ester 20 kDa (mPEG-6AC-NHS):

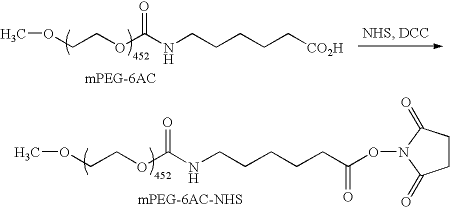

N-6-[methoxypoly(ethyleneglycol)]-oxo-aminocaproic acid (55.16 g, 2.74 mmol) was dissolved in CH₂Cl₂ (200 mL) and cooled in an ice bath under an atmosphere of Ar. N-Hydroxysuccinimide (631 mg, 5.48 mmol) and DCC (1.13 g, 5.48 mmol, in CH₂Cl₂ (5 mL)) were added and the resulting mixture was stirred at room temperature for 17 h. The reaction mixture was filtered to remove the crystals which had formed and the filtrate was concentrated under vacuum. The resulting white solid was washed with ether (600 mL), filtered and covered with isopropanol (800 mL). The suspension was warmed to 50° C. until the solid completely dissolved after which the solution was cooled in an ice bath for 2 h. The suspension was filtered, and the solid redissolved in CH₂Cl₂ (160 mL). Ether (1600 mL) was added and the resulting white precipitate was collected by filtration and dried under vacuum to give mPEG-6AC-NHS (53.2 g, 2.63 mmol, 96%) as a white solid.

¹H NMR (200 MHz, DMSO-d₆): δ_{H} 7.09-7.29 (m, 1 H), 3.98-4.09 (m, 1 H), 3.11-3.93 (m, 1806 H), 3.25 (s, 3 H), 2.91-3.04 (m, 2 H), 2.82 (s, 4 H), 2.65 (t, J=7.59, 2 H), 1.51-1.67 (m, 2 H), 1.26-1.49 (m, 4 H). IR; nujol, cm⁻¹, 1813, 1783, 1740, 1713, 1148, 1109, 1061.

N-5-[Methoxypoly(ethyleneglycol)]-oxo-aminovaleric acid 5 kDa (mPEG-5AV):

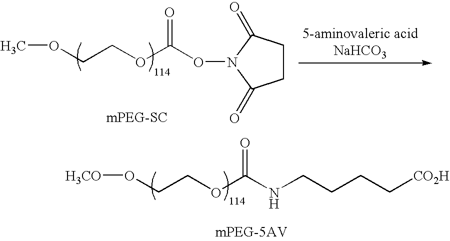

5-Aminovaleric acid (91 mg, 0.778 mmol) and NaHCO₃ (65 mg, 0.778 mmol) were dissolved in a mixture of H₂O (14 mL) and ethanol (6 mL). Methoxypoly(ethyleneglycol) succinimidyl carbonate (2.0 g, 0.39 mmol) was added and the mixture was stirred at room temperature for 2 h. HCl (1 N) was added until the solution reached pH 3. The resulting solution was extracted with CH₂Cl₂ (3×50 mL) and the combined organic extracts were dried (Na₂SO₄), filtered and concentrated under vacuum to give mPEG-5-AV acid (1.66 g, 83%) as a white solid.

¹H NMR (200 MHz, DMSO-d₆): δ_{H} 7.14-7.29 (m, 1 H), 4.05 (m, 2 H), 3.55 (s, 452 H), 3.25 (s, 3 H), 2.95 (q, J=5.94Hz, 2 H), 2.20 (t, J=7.32, 2 H), 1.25-1.6 (m, 4H). IR; nujol, cm⁻¹, 1716, 1103, 1099.

N-5-[Methoxypoly(ethyleneglycol)]-oxo-aminovalerate N-hydroxysuccinimide ester(mPEG-5AV-NHS):

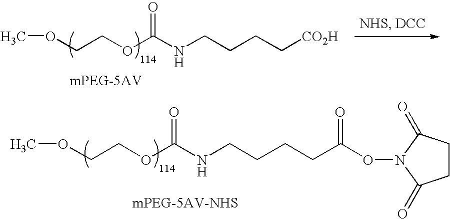

N-5-[methoxypoly(ethyleneglycol)]-oxo-aminovaleric acid (1.66 g, 0.323 mmol) was dissolved in CH₂Cl₂ (8.5 mL) and cooled in an ice bath under an atmosphere of Ar. N-Hydroxysuccinimide (74 mg, 0.65 mmol) was added, and DCC (133 mg, 0.65 mmol, in CH₂Cl₂ (0.5 mL) was added. The resulting mixture was stirred at room temperature for 17 h. The reaction mixture was filtered to remove the crystals which had formed and the filtrate was concentrated under vacuum. The resulting white solid was recrystallized with isopropanol (10 mL). The solid was redissolved in CH₂Cl₂ (3 mL). Ether (50 mL) was added and the resulting white precipitate was collected by filtration and dried under vacuum to give mPEG-5AV-NHS (1.23 g, 0.24 mmol, 72%) as a white solid.

¹H NMR (200 MHz, DMSO-d₆): δ_{H} 7.09-7.29 (m, 1 H), 3.98-4.09 (m, 1 H), 3.55 (s, 452 H), 3.25 (s, 3 H), 3.0 (q, J=5.78, 2H), 2.83 (s, 4 H), 2.70 (t, J=6.31 Hz, 2 H), 1.42-1.80 (m, 4 H). IR; nujol, cm⁻¹, 1813, 1783, 1740, 1713, 1148, 1109, 1061.

N-4-[Methoxypoly(ethyleneglycol)]-oxo-aminobutyric acid 5 kDa (mPEG-4AB):

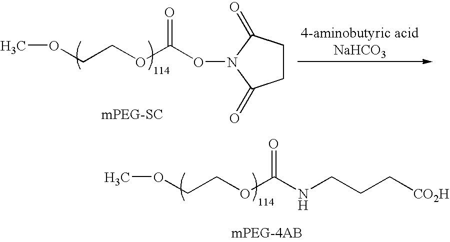

4-Aminobutyric acid (80 mg, 0.778 mmol) and NaHCO₃ (65 mg, 0.778 mmol) were dissolved in a mixture of H₂O (14 mL) and ethanol (6 mL). Methoxypoly(ethyleneglycol) succinimidyl carbonate (2.0 g, 0.39 mmol) was added and the mixture was stirred at room temperature for 2 h. HCl (1 N) was added until the solution reached pH 3. The resulting solution was extracted with CH₂Cl₂ (3×50 mL) and the combined organic extracts were dried (Na₂SO₄), filtered and concentrated under vacuum to give mPEG-4-AB acid (1.77 g, 88%) as a white solid.

¹H NMR (200 MHz, DMSO-d₆): δ_{H} 7.14-7.29 (m, 1 H), 4.05 (t, J=4.44, 2 H), 3.55 (s, 452 H), 3.25 (s, 3 H), 3.0 (q, J=6.31 Hz, 2 H), 2.22 (t, J=7.4, 2 H), 1.62 (pent, J=7.4 Hz, 2H). IR; nujol, cm⁻¹, 1716, 1103, 1099.

N-4-[Methoxypoly(ethyleneglycol)]-oxo-aminobutyrate N-hydroxysuccinimide ester 5 kDa (mPEG-4AB-NHS):

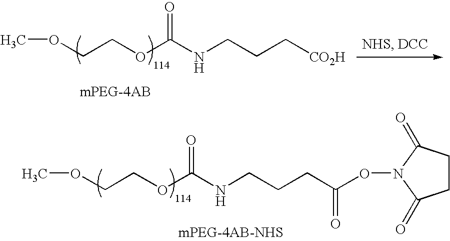

N-4-[methoxypoly(ethyleneglycol)]-oxo-aminobutyric acid (1.77 g, 0.345 mmol) was dissolved in CH₂Cl₂ (8.2 mL) and cooled in an ice bath under an atmosphere of Ar. N-Hydroxysuccinimide (79 mg, 0.69 mmol) was added, and DCC (142 mg, 0.69 mmol, in CH₂Cl₂ (0.5 mL) was added. The resulting mixture was stirred at room temperature for 17 h. The reaction mixture was filtered to remove the crystals which had formed and the filtrate was concentrated under vacuum. The resulting white solid was recrystallized with isopropanol (15 mL). The solid was redissolved in CH₂Cl₂ (5 mL). Ether (100 mL) was added and the resulting white precipitate was collected by filtration and dried under vacuum to give mPEG-4AB-NHS (1.35 g, 0.26 mmol, 75%) as a white solid.

¹H NMR (200 MHz, DMSO-d₆): δ_{H} 7.09-7.29 (m, 1 H), 3.98-4.09 (m, 1 H), 3.55 (s, 452 H), 3.25 (s, 3 H), 2.98 (q, J=5.85, 2H), 2.83 (s, 4 H), 2.68 (t, J=7.59, 2 H), 1.52-1.72(m,2H). IR; nujol, cm⁻¹, 1813, 1783, 1740, 1713, 1148, 1109, 1061.

The above reactions can be done starting with PEG in place of the mPEG to make the succinimidyl carbonates, so that the succinimidyl carbonate will be at both ends of the compound (di-succinimidyl carbonate PEG), and subsequently the oxo-amino acid N-hydroxysuccinimide ester will be at both ends of the compound (di-6AC-NHS-PEG).

### EXAMPLE 22

### Synthesis of Various mPEG-6AC Esters and Thio Esters

The following procedures can be followed to make a variety of active mPEG-6AC derivatives. As per Example 1, the mPEG-6AC could also be prepared as di-6AC-PEG by making the di-succinimidyl carbonate PEG from PEG instead of mPEG. The resulting compounds would have the oxo-6-aminocaproic acid esters and thiols at both ends.

Procedure A:

N-6-[methoxypoly(ethyleneglycol)]-oxo-aminocaproic acid (1 g, 49.7 μmol) was dissolved in CH₂Cl₂ (10 mL). Add EDCI, (1.5 eq.), DMAP (0.1 eq.) and the alcohol (10 eq.) or thiol (10 eq.), and stir at room temperature under an atmosphere of N₂ until TLC (reverse phase, C8, CH₃OH/H₂O 4/1, v/v) indicated complete conversion to a less polar product. Ether (100 mL) was added and the resulting suspension was cooled to 0° C. for 1 h. The suspension was filtered and the white solid recrystallized from isopropanol (20 mL). The solid was redissolved in CH₂Cl₂ (10 mL) and ether (100 mL) was added. The resulting solution was cooled to 0° C. for 1 h after which the white precipitate was collected by filtration and dried under vacuum.

Procedure B:

N-6-[methoxypoly(ethyleneglycol)]-oxo-aminocaproic acid (1 g, 49.7 μmol) was dissolved in CH₃CN (10 mL). Add PyBOP (1 eq.), HOBT (1 eq.), Et₃N (2 eq.) and the alcohol (5 eq.) or thiol (5 eq.), and stir at room temperature under an atmosphere of N₂ until TLC (reverse phase, C8, CH₃OH/H₂O 4/1, v/v) indicated complete conversion to a less polar product. Ether (100 mL) was added and the resulting suspension was cooled to 0° C. for 1 h. The suspension was filtered and the white solid recrystallized from isopropanol (20 mL). The solid was redissolved in CH₂Cl₂ (10 mL) and ether (100 mL) was added. The resulting solution was cooled to 0° C. for 1 h after which the white precipitate was collected by filtration and dried under vacuum.

Procedure C:

N-6-[methoxypoly(ethyleneglycol)]-oxo-aminocaproic acid (1 g, 49.7 μmol) was dissolved in CH₃CN (10 mL). Add HBTU (1 eq.), Et₃N (2 eq.) and the alcohol (5 eq.) or thiol (5 eq.), and stir at room temperature under an atmosphere of N₂ until TLC (reverse phase, C8, CH₃OH/H₂O 4/1, v/v) indicated complete conversion to a less polar product. Ether (100 mL) was added and the resulting suspension was cooled to 0° C. for 1 h. The suspension was filtered and the white solid recrystallized from isopropanol (20 mL). The solid was redissolved in CH₂Cl₂ (10 mL) and ether (100 mL) was added. The resulting solution was cooled to 0° C. for 1 h after which the white precipitate was collected by filtration and dried under vacuum.

Abbreviations:

EDCI 1-[3-(Dimethylamino)propyl]-3-ethylcarbodimide

HOBT 1-Hydroxybenzotriazole

PyBOP Benzotriazole-1-yl-oxy-tris-pyrrolidino-phosphonium hexafluorophosphate

HBTU 2-(1H-Benzotriazole-1-yl)-1,1,3,3-tetramethyluronium hexafluorophosphate

PyBOP and HBTU were purchased from Calbiochem (San Diego, Calif.). All other chemicals are from Aldrich.

N-6-[Methoxypoly(ethyleneglycol)]-oxo-aminocaproate pentafluorophenyl ester 20 kDa (mPEG-6AC-PFP):

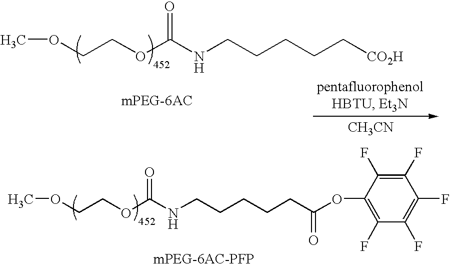

Prepared using procedure C. Obtained 953 mg, 47.0 μmol, 87% as a white solid. ¹H NMR (200 MHz, DMSO-d₆): δ_{H} 7.17-7.30 (m, 1 H), 3.99-4.10 (m, 2H), 3.11-3.93 (m, 1806 H), 3.25 (s, 3 H), 2.89-3.04 (m, 2 H), 2.80 (m, 2 H), 1.57-1.79 (m, 2 H), 1.26-1.51 (m, 4 H). IR; nujol, cm⁻¹, 1788, 1719, 1674, 1105, 1061.

N-6-[Methoxypoly(ethyleneglycol)]-oxo-aminocaproate 2,2,2-trifluoroethyl ester 20 kDa (mPEG-6AC-TFE):

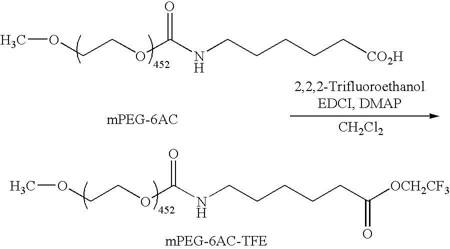

Prepared using procedure A. Obtained 957 mg, 47.4 μmol, 82% as a white solid. ¹H NMR (200 MHz, DMSO-d₆): δ_{H} 7.19-7.31 (m, 1 H), 4.65-4.84 (m, 2 H), 3.97-4.10 (m, 2 H), 3.18-3.95 (m, 1809 H), 2.87-3.06 (m, 2 H), 2.37-2.48 (m, 2 H), 1.14-1.62 (m, 6 H). IR; nujol, cm⁻¹, 1756, 1717, 1641, 1148, 1109, 1057.

N-6-[Methoxypoly(ethyleneglycol)]-oxo-aminocaproate ethyl ester 20 kDa (mPEG-6AC-OEt):

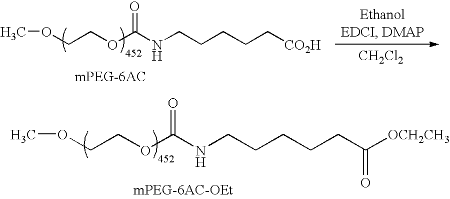

Prepared using procedure A. Obtained 940 mg, 46.7 μmol, 79% as a white solid. ¹H NMR (200 MHz, DMSO-d₆): δ_{H} 7.05-7.26 (m, 1 H), 4.01-4.19 (m, 2 H), 3.11-4.01 (m, 1809 H), 2.92-3.04 (m, 2 H), 2.20-2.36 (m, 2 H), 1.10-1.65 (m, 9 H),. IR; nujol, cm⁻¹, 1720, 1141, 1111, 1060.

N-6-[Methoxypoly(ethyleneglycol)]-oxo-aminocaproate pentafluorobenzenethio ester 20 kDa (mPEG-6AC-PFT):

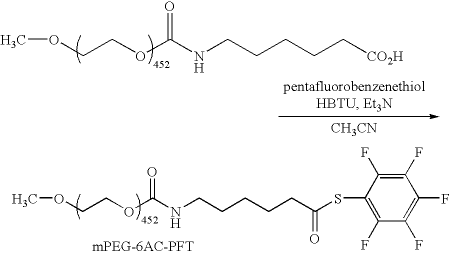

Prepared using procedure C. Obtained 0.84 g, 41.4 μmol, 89% as a white solid. ¹H NMR (200 MHz, DMSO-d₆): δ_{H} 7.13-7.25 (m, 1 H), 3.99-4.12 (m, 2 H), 3.08-3.95 (m, 1809 H), 2.79-3.07 (m, 4 H), 1.19-1.71 (m, 6 H). IR; nujol, cm⁻¹, 1719, 1640, 1145, 1111, 1061.

N-6-[Methoxypoly(ethyleneglycol)]-oxo-aminocaproate 2,3,5,6-tetrafluorobenzene thio ester 20 kDa (mPEG-6AC-TFT):

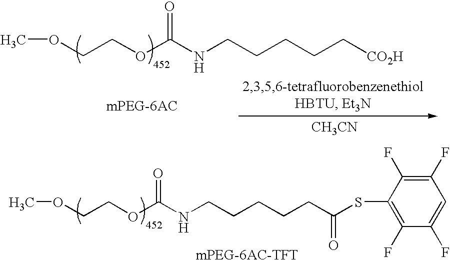

Prepared using procedure C. Obtained 1.10 g, 54.2 μmol, 98% as a white solid. ¹H NMR (200 MHz, DMSO-d₆): δ_{H} 8.06-8.31 (m, 1 H), 7.13-7.25 (m, 1 H), 3.99-4.11 (m, 2 H), 3.08-3.95 (m, 1809 H), 3.26 (s, 3 H), 2.77-3.07 (m, 4 H), 1.53-1.75 (m, 2 H), 1.22-1.53 (m, 4 H). IR; nujol, cm⁻¹, 1720, 1633, 1148, 1110, 1061.

N-6-[Methoxypoly(ethyleneglycol)]-oxo-aminocaproate 4-fluorobenzenethio ester 20 kDa (mPEG-6AC-4FTP):

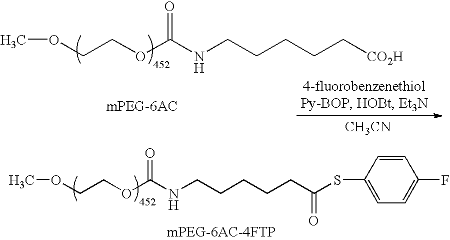

Prepared using procedure B. Obtained 0.94 g, 54.2 μmol, 88% as a white solid. ¹H NMR (200 MHz, DMSO-d₆): δ_{H} 7.25-7.59 (m, 3 H), 3.97-4.11 (m, 2 H), 3.07-3.96 (m, 1809 H), 2.90-3.03 (m, 2 H), 2.64-2.77 (m, 2 H), 1.53-1.76 (m, 2 H), 1.25-1.49 (m, 4 H). IR; nujol, cm⁻¹, 1710, 16.76, 1633, 1147, 1108, 1061.

N-6-[Methoxypoly(ethyleneglycol)]-oxo-aminocaproate 2,2,2-trifluoroethylthio ester 20 kDa (mPEG-6AC-TFET):

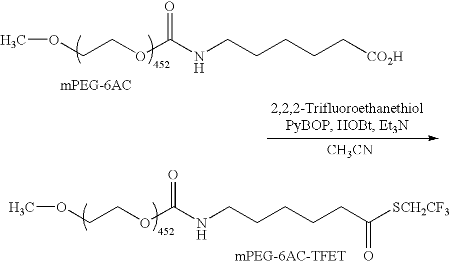

Prepared using procedure B. Obtained 1.04 g, 51.4 μmol, 86% as a white solid. ¹H NMR (200 MHz, DMSO-d₆): δ_{H} 7.07-7.25 (m, 1 H), 3.99-4.10 (m, 2 H), 3.09-3.94 (m, 1809 H), 2.89-3.00 (m, 2 H), 2.62-2.77 (m, 2 H), 1.51-1.74 (m, 2 H), 1.19-1.48 (m, 4 H). IR; nujol, cm⁻¹, 1722, 1142, 1108, 1062.

N-6-[Methoxypoly(ethyleneglycol)]-oxo-aminocaproate ethylthio ester 20 kDa (mPEG-6AC-SEt):

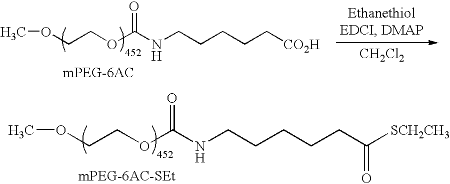

Prepared using procedure A. Obtained 0.86 g, 42.6 μmol, 85% as a white solid. ¹H NMR (200 MHz, DMSO-d₆): δ_{H} 7.03-7.30 (m, 1 H), 3.98-4.11 (m, 2 H), 3.04-3.97 (m, 1809 H), 2.78-3.03 (m, 4 H), 1.06-1.78 (m, 9 H). IR; nujol, cm⁻¹, 1785, 1718, 1646, 1148, 1109, 1061.

### EXAMPLE 23

### Antigen Masking of Red Cells Reacted with mPEG-6AC, 5AV or 4AB-NHS and mPEG-6AC-PFP

A unit of ABO matched whole blood (Sacramento Blood Center, CA) was leukofiltered according to standard blood banking methods. The RBC were centrifuged at 4° C. at 4100×g for 6 minutes and the plasma was removed to give a red cell concentrate. The red cell concentrate was washed with an equal volume of reaction buffer, centrifuged as above, the supernatant removed and the wash repeated. The washed red cell concentrate was reacted with an activated mPEG by dissolving the mPEG in reaction buffer and adding it to the red cell concentrate to a hematocrit of approximately 40% and the desired mPEG concentration in the extracellular volume. The reaction buffer was either 150 mM CHES pH 9 with 100 mM dextrose and 5 mM L-carnitine (CHES-GC) or 150 mM CHES pH 9 with 50 mM NaCl (CHES-Na). The reaction mixture was incubated for 2 hours at room temperature and an equal volume of wash buffer was added, the samples centrifuged as above, the supernatant removed and the wash repeated. The wash buffer was either 150 mM Na₂HPO₄ pH 7, 50 mM NaCl (PBS), or 150 mM Na₂HPO₄ pH 7, 100 mM dextrose, 5 mM L-carnitine (PB-GC). The final red cell solution is stored in an approximately equal volume of Erythrosol and assayed for anti-A antibody binding as per Example 10. The results are found in Table 9. All compounds showed effective masking of the red cell antigens.

TABLE 9
Modification of red cells with various activated mPEGs as measured by
anti-type antibody binding.

| | Reaction solution | | Post reaction wash | | [mPEG] | % anti-A |
|---|---|---|---|---|---|---|
| mPEG Compound | 150 mM CHES pH 9 | | 150 mM Na₂HPO₄ pH 7 | | mM | binding |
| 5 kDa 4AB-NHS | 50 | mM NaCl | 50 | mM NaCl | 22 | 1.0 |
| 5 kDa 5AV-NHS | 50 | mM NaCl | 50 | mM NaCl | 22 | 1.2 |
| 5 kDa 6AC-NHS | 50 | mM NaCl | 50 | mM NaCl | 22 | 1.0 |
| 20 kDa 6AC-PFP | 100 | mM dextrose, | 100 | mM dextrose, | 5 | 0.4 |
| | 5 | mM L-carnitine | 5 | mM L-carnitine | | |

### EXAMPLE 24

### Adjustment of the Extracellular pH with the Use of an Ion Exchange Resin

In order to demonstrate the ability to use an ion exchange resin to control the pH of the extracellular domain of RBC, a 30 mL sample of whole blodd collected with CPDA-1 anti coagulant was added to a 50 mL tube. A 1 g amount of IRA 400 OH strong anion exchange resin (Supelco, Bellefonte, Pa.) was added to the whole blood and the suspension was rmixed by inverting gently five times. The pH was measured at room temperature as a function of time using a Beckman Epoxy Calomel electrode. The pH stabilized at 8.11 after 15 minutes. A second 1 g amount of resin was added and the pH monitored as a function of time. The pH stabilized at 9.47 after 5 minutes. The results are found in Table 10. During the incubation with the resin, minor hemolysis was observed. The hematocrit of the samples did not change during incubation, indicating that the resin did not adsorb any liquid. The sample was stored overnight and the pH was 9.17, indicating that the high pH is maintained for long periods. This suggests that the resin would be suitable for providing the desired pH for reaction of activated PEG compounds.

TABLE 10
pH over time on incubation of whole blood with IRA 400 OH resin.

| | | pH prior | pH after | | |
|---|---|---|---|---|---|
| Time after | | to first | addition of | pH prior to | pH after addition |
| addition | | addition | first gram | second addition | of second gram |
| of resin | | of resin | of resin | of resin | of resin |
| 0 | | 7.19 | — | 8.12 | — |
| 0.5 | minutes | | 7.70 | | 8.66 |
| 2 | minutes | | 7.90 | | 9.39 |
| 3 | minutes | | 8.02 | | — |
| 5 | minutes | | 8.08 | | 9.47 |
| 10 | minutes | | 8.11 | | 9.47 |
| 15 | minutes | | 8.12 | | 9.47 |
| 20 | hours | | — | | 9.17 |

We claim:

1. A method of preparing a red cell composition comprising:
a) providing i) a red cell concentrate, ii) an activated polyethylene glycol compound, and iii) a reaction solution comprising a buffer at a concentration of approximately 75-350 mM and at a pH of approximately 8-10;
b) washing the red cell concentrate with the reaction solution to provide a washed red cell concentrate;
c) mixing the washed red cell concentrate with the reaction solution and the activated polyethylene glycol compound to provide a reaction mixture; and
d) incubating the reaction mixture so that the activated polyethylene glycol compound covalently binds to the red cell surface.

2. The method of claim 1, wherein the buffer concentration is 100 to 200 mM.

3. The method of claim 2, wherein the buffer concentration is 150 mM.

4. The method of claim 1, wherein the buffer has a pKa of 8.5 to 9.5.

5. The method of claim 1, wherein the buffer is at a pH of 8.5-9.5.

6. The method of claim 5, wherein the buffer is at a pH of 8.5-9.

7. The method of claim 6, wherein the buffer is at a pH of about 9.

8. The method of claim 1, wherein the buffer is CHES.

9. The method of claim 7, wherein the buffer is CHES.

10. The method of claim 9, wherein the CHES is at a concentration of 150 mM.

11. The method of claim 1, wherein the covalent binding of the polyethylene glycol to the red cell results in a red cell that does not bind to any antibodies to red cell antigens when tested in a standard agglutination test.

12. The method of claim 1, wherein the covalent binding of the polyethylene glycol to the red cell is such that the binding of fluorescent anti-type antibody to the modified red cells as measured by FACScan is less than 30% of the binding to the same source of red cells that has not been modified.

13. The method of claim 12, wherein the binding of anti-type antibody to the modified red cells is less than 10% of the binding of the unmodified red cells.

14. The method of claim 13, wherein there is essentially no binding of anti-type antibody to the modified red cells.

15. The method of claim 1, wherein the covalent binding of the polyethylene glycol to the red cell results in a red cell that does not bind to any antibodies to minor red cell antigens when tested in a standard agglutination test for minor antigens.

16. A composition comprising red cells prepared according to claim 1.

17. A composition comprising red cells prepared according to claim 4.

18. A composition comprising red cells prepared according to claim 5.

19. A composition comprising red cells prepared according to claim 8.

20. A composition comprising red cells prepared according to claim 11.

21. A composition comprising red cells prepared according to claim 12.

22. A composition comprising red cells prepared according to claim 13.
